(19) United States
(12) Patent Application Publication
(10) Pub. No.: US 2025/0312529 A1
(43) Pub. Date: Oct. 9, 2025

(54) DUAL-SENSOR DIFFERENTIAL PRESSURE TRANSDUCER SYSTEM

(71) Applicant: Becton, Dickinson and Company, Franklin Lakes, NJ (US)

(72) Inventors: John Randolph Dewing, Costa Mesa, CA (US); Lauren Ashley Cantu, Laguna Niguel, CA (US)

(21) Appl. No.: 19/244,184

(22) Filed: Jun. 20, 2025

Related U.S. Application Data
(63) Continuation of application No. PCT/US2023/085016, filed on Dec. 20, 2023.
(60) Provisional application No. 63/476,363, filed on Dec. 20, 2022.

(51) Int. Cl. A61M 5/14; A61M 39/22; G01F 1/36

(52) U.S. Cl. CPC A61M 5/1414; A61M 39/223; G01F 1/363; A61M 2005/1403; A61M 2202/0007; A61M 2202/04; A61M 2205/3344

(57) ABSTRACT

A dual differential pressure transducer assembly includes a flowpath element forming a fluid channel from an intravenous (IV) fluid inlet to an IV fluid outlet and defining a centerline axis of the assembly. First and second sensor cavities are disposed alongside the fluid channel, in parallel and separately connected to the fluid channel via first and second cutouts through the flowpath element, transverse to the centerline axis. First and second differential pressure sensors abut respective cavities, and are exposed to the fluid channel via the cutouts. Separate signal conductors are electrically connected to the first and second differential pressure sensors, and sheathed within a common connector cable.

### CROSS-REFERENCE TO RELATED APPLICATION(S)

This application is a continuation of PCT Application No. PCT/US2023/085016, filed Dec. 20, 2023, and entitled “DUAL-SENSOR DIFFERENTIAL PRESSURE TRANSDUCER SYSTEM,” which in turn claims the benefit of U.S. Provisional Application No. 63/476,363, filed Dec. 20, 2022, and entitled “DUAL-SENSOR DIFFERENTIAL PRESSURE TRANSDUCER SYSTEM,” the disclosure of which is hereby incorporated by reference in its entirety.

### BACKGROUND

This disclosure relates generally to apparatus for intravenous (IV) fluid delivery. More specifically, the present disclosure concerns a differential pressure transducer (DPT) system used to monitor fluid pressures in IV fluid delivery systems.

DPT systems are commonly included between IV bags and needles to sense rates of fluid flow, and are communicatively coupled to patient monitors and other electronics, e.g. to regulate and generate records of fluid delivery and to trigger dosage warnings. Fluid flows through the DPT system, which provides an electrical signal reflecting differential pressure to an attached patient monitor via an electrical connection.

DPT systems for IV fluid monitoring are sterile elements disposed along an IV line, typically at a location close to or even secured to a patient, e.g. taped to the patient's arm. As the patent is transferred from one location (e.g. an ambulance) to another (e.g. a hospital), the DPT system may stay with the patient but be fluidly connected to multiple IV bags and provide signal outputs used by multiple different patient monitoring devices or systems in different environments. To ensure proper hygiene, DPT systems are not generally reused. For these reasons, DPT systems are typically disposable devices configured to connect to and provide sensor signals to a wide variety of electronic systems for patient monitoring.

### SUMMARY

In one illustrative example, this disclosure presents a dual differential pressure transducer assembly includes a flowpath element forming a fluid channel from an intravenous (IV) fluid inlet to an IV fluid outlet and defining a centerline axis of the assembly. First and second sensor cavities are disposed alongside the fluid channel, in parallel and separately connected to the fluid channel via first and second cutouts through the flowpath element, transverse to the centerline axis. First and second differential pressure sensors abut respective cavities, and are exposed to the fluid channel via the cutouts. Separate signal conductors are electrically connected to the first and second differential pressure sensors, and sheathed within a common connector cable.

In another illustrative example, this disclosure presents a differential pressure transducer assembly that includes a fluid channel for IV fluid, first and second differential pressure sensors disposed to generate respective signals indicative of fluid flow through the fluid channel, and first and second pluralities of signal conductors electrically respectively connected to the first and second differential pressure sensors. The conductors are carried within a common connector cable to a connector plug having separate pluralities of pins contacting the first and second signal conductors. A wire guide separates the first from the second plurality of signal conductors, and guides each to its respective pin.

In still another illustrative example, this disclosure presents a sensor signal connector configured to separately carry sensor signals from a first sensor and a second sensor. The sensor signal connector includes separate first and second pluralities of signal conductors electrically connected to the first and second sensors, respectively. A common connector cable surrounds both pluralities of signal conductors, and a wire guide with opposite first and second sides is disposed at an end of the connector cable. The wire guide retains exposed ends of the first plurality of signal conductors at connection locations on the first end, and retains exposed ends of the second plurality of signal conductors on the second end. A plurality of pins are each attached to one of the first or second pluralities of signal conductors and secured in the wire guide. A modular plug defines an outer form factor of the sensor signal connector securable in a sensor receptacle and surrounds the wire guide, thereby securing the first and second pluralities of signal conductors to the wire guide and exposing the plurality of pins.

In yet another illustrative example, this disclosure presents a multi-function signal connector configured to receive both analog and digital sensor signals. The multi-function signal connector includes an oval socket disposed about a receptacle axis and defining a receptacle space, and a rigid contact support disposed within the oval socket. The rigid contact support includes a top shelf and a bottom shelf. A first plurality of electrical contacts is disposed between the top shelf and the oval socket and angled from the top shelf towards the bottom shelf, while a second plurality of electrical contacts disposed between the bottom shelf and the oval socket and angled from the bottom shelf towards the top shelf.

In yet another illustrative example, this disclosure presents a snap fit connection between a flowpath element and a structural housing of the DPT assembly. The snap fit connection includes multiple protrusions extending from the flowpath element or the structural housing. The protrusions insert into receptacles of the adjacent component. Protrusion geometry such as length, width, and thickness of the protrusions and, in some cases, stiffening ribs permit adjustment of the insertion force and extraction force along a single line of action.

In yet another illustrative example, this disclosure presents a restraint of DPT assembly internal components that does not require adhesive. The restraint includes cantilevered supports extending from a front cover or a rear cover of the DPT assembly. The cantilevered supports elastically engage a structural housing biasing a flowpath element into engagement with a tapered stopcock bore via the structural housing. The stopcock includes a collar, which may be divided into two or more segments, that engages an equal number of retaining slots of a rear cover pedestal to oppose the bias imposed by the cantilevered supports. Once fully assembled, the cantilevered supports compress the DPT/Stopcock subassembly collar against its retaining slot to keep the joint under constant compression, maintaining an airtight seal.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a schematic view of a patient monitoring system with a single monitor and a differential pressure transducer (DPT) system.

FIG. 2 is a schematic view of a patient monitoring system with multiple connected devices, including the DPT system and a multi-signal pressure cable connected thereto.

FIG. 3 is a perspective view of the DPT system with a hybrid connector.

FIG. 4 is a front plan view of the DPT system of FIG. 3, including a DPT sub-assembly with a fluid channel with a stopcock.

FIGS. 5 and 6 are perspective and cross-sectional perspective views, respectively, of components of the DPT sub-assembly of FIG. 4 disposed along an axis of the fluid channel.

FIG. 7 is an exploded view of components of the DPT sub-assembly of FIGS. 4-6 including a rear cover, a structural assembly, a flowpath element, and a stopcock.

FIG. 8A and FIG. 8B are exploded views of a flowpath element and a structural housing.

FIG. 8C is a cross-sectional perspective view through the structural housing that illustrates features of receptacles.

FIG. 8D is a cross-sectional perspective view through the flowpath element and the structural housing that illustrates protrusions and receptacles.

FIG. 8E is a cross-sectional view depicting an interface of the flowpath element, the structural housing, and a connector cable.

FIG. 9A is a cross-sectional view extending longitudinally through the DPT subassembly that illustrates the flowpath element, the structural housing, and a stopcock.

FIGS. 9B and 9C are interior perspective views of a front cover depicting exemplary cantilevered supports.

FIG. 9D is a perspective detail view of an interface among the flowpath element, a stopcock, and a support pedestal of a rear cover.

FIG. 9E is a cross-sectional view taken through a stopcock and a rear cover pedestal depicting features of a hard stop.

FIGS. 10A and 10B are broken-away plan and perspective views, respectively, of the DPT sub-assembly of FIG. 4 illustrating separate analog and digital sensors with dedicated sensor connectors.

FIG. 11 is a plan view of the DPT sub-assembly as shown in FIG. 5, with sensors removed and sensor cavities visible.

FIG. 12 is a perspective view of the DPT sub-assembly of FIGS. 10A, 10B, and 11 along a centerline axis of the fluid channel, illustrating a channel aperture of a cutout to a sensor.

FIG. 13 is a cross-sectional perspective view of the DPT sub-assembly of FIGS. 10A, 10B, 11, and 12 illustrating the positioning of both sensors and respective cutouts relative to the fluid channel.

FIG. 14 is perspective view of the signal connector of FIG. 3.

FIGS. 15A and 15B are close-up perspective views of a plug of the signal connector of FIG. 14.

FIG. 16 is a perspective view illustrating internal wires, pins, and a wire guide of the plug of FIGS. 15A and 15B.

FIG. 17 is a perspective view of the components of FIG. 16 enclosed in a modular plug.

FIG. 18 is a perspective view of the multi-signal pressure cable of FIG. 2

FIG. 19 is a perspective view of a receptacle end of the multi-signal pressure cable of FIG. 18, with outer housing of the multi-signal pressure cable omitted.

FIGS. 20A and 20B are perspective views into the multi-signal pressure cable of FIG. 19.

FIG. 21 is a perspective view of the multi-signal pressure cable of FIG. 20, with overmold omitted to expose an oval socket.

FIGS. 22A and 22B are perspective views of the multi-signal pressure cable of FIG. 21, with the overmold omitted to expose electrical contacts and a contact support.

FIG. 23 is a perspective view of the multi-signal pressure cable of FIGS. 22A and 22B, with the contact support omitted to illustrate shapes of the electrical contacts.

FIG. 24 is a cross-sectional view through the oval socket and contact support of FIGS. 21 and 22A.

FIGS. 25A and 25B are perspective and cross-sectional perspective views, respectively, of a signal connector of the DPT system of FIG. 3, connected to a receptacle.

While the above-identified figures set forth one or more examples discussed with the present disclosure, variations and permutations of these examples are also contemplated, as noted in the discussion. In all cases, this disclosure presents the invention by way of representation and not limitation. It should also be understood that numerous other modifications can be devised by those skilled in the art from the present disclosure, which accordingly fall within the scope and spirit of the principles of this invention. The figures may not be drawn to scale, and applications and examples of the present invention may include features and components not specifically shown in the drawings.

### DETAILED DESCRIPTION

This disclosure presents a dual- or multi-sensor differential pressure transducer system for use in IV fluid delivery. The examples provided below present two sensors—e.g. one analog and one digital sensor—disposed in parallel at a common axial location along an IV fluid flowpath. These sensors are contained and supported within a common assembly including a flowpath element with a fluid channel defining the IV fluid flowpath, sandwiched between but extending axially beyond front and rear covers. The front and rear covers and flowpath element cooperate to axially retain a stopcock in a sealed fluid connection with a downstream end of the flowpath element, while a structural support disposed between the DPT housing and the flowpath element cooperates with the flowpath element to retain both sensors against respective sensor cavities in fluid communication with the fluid channel.

The DPT system disclosed herein includes a DPT sub-assembly with separate compact digital and analog pressure sensors disposed in respective cavities laterally offset from and axially aligned with each other, and connected to a fluid channel of the sub-assembly via axially symmetrically spaced cutouts. The DPT sub-assembly connects to a patient monitor via a hybrid signal connector incorporating separate analog and digital signal contacts in a single signal plug received by a matching hybrid receptacle. The DPT system includes a signal connector wherein sets of electrical conductors contacting the digital and analog sensors are enclosed within a common conductor cable, and carry parallel sensor signals from the sensors to the shared connector plug. This shared connector plug includes separate contacts for each sensor, e.g. one set of digital contacts and one set of analog contacts, within a common form factor receivable at a digital connector socket, an analog connector socket, or a hybrid multi-signal receptacle.

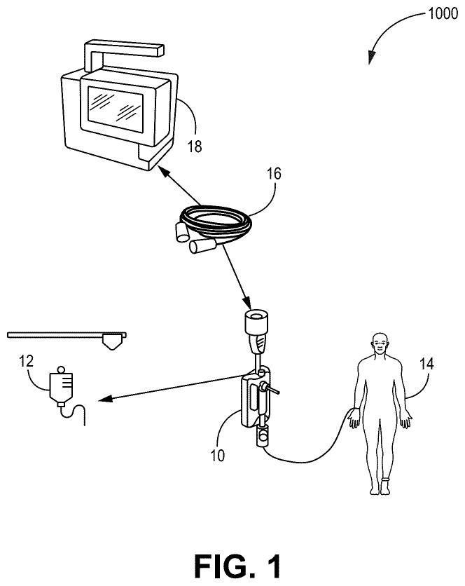

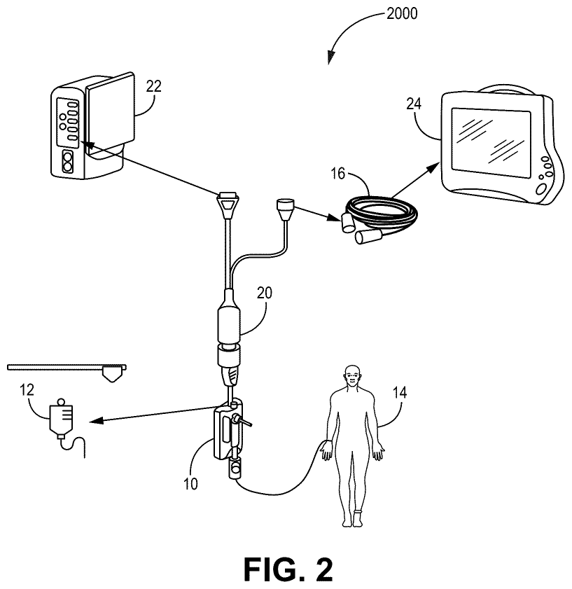

FIGS. 1 and 2 provide schematic views of two example patient monitoring systems incorporating DPT system 10. FIG. 1 depicts patient monitoring system 1000, while FIG. 2 depicts related patient monitoring system 2000. Patient monitoring systems 1000 and 2000 are non-exclusive examples of expected systems in which DPT system 10 can be used, and are provided to illustrate different use cases of DPT system 10. Several aspects of FIGS. 1 and 2 are hereinafter described together. FIGS. 1 and 2 both depict IV fluid source 12, patient 14, and patient monitor cable 16. FIG. 1 additionally illustrates a single transport monitor 18, while FIG. 2 depicts multi-signal pressure cable 20 providing a signal connection to both primary monitor 22 and secondary monitor 24.

Patient monitoring systems 1000 and 2000 are used to track vital information and/or treatment information of patient 14 while patient 14 receives medical care including the provision of IV fluid. In one illustrative example, patient monitoring system 1000 can be a system for use in a transport environment, e.g. for a mobile device used during transfer of a patent from emergency or operating room to an intensive care unit. By contrast, patient monitoring system 1000 can be a system in a static environment such as a hospital intensive care unit or operating room. More generally, however, patient monitoring systems 1000 and 2000 can represent any separate systems between which a patient with a single DPT system 10 may be transferred, including multiple vehicles or separate static environments.

Fluid source 12 is schematically illustrated as an IV bag and can more generally be any sort of IV fluid supply. IV fluid from fluid source 12 can, for example, be delivered to patient 14 by an IV shunt or needle inserted in an arm of patient 14. More generally, fluid source 12 can be any sort of reservoir capable of providing a continuous safe supply of IV fluid to patient 14 via an IV attachment. DPT system 10 is a transducer system disposed between fluid source 12 and patient 14 to sense differential fluid pressure, and thereby fluid flow, therebetween. DPT system 10 generates sensor signals reflecting differential pressure of the IV fluid. These sensor signals are received and processed by patient monitoring equipment for a variety of purposes, including but not limited to the metering of medication, tracking of IV fluid dispensing, and triggering of dosage warnings. As shown in later figures and discussed in greater detail below, DPT system 10 includes a fluid channel through which IV fluid flows from fluid source 12 toward patient 14, and an electrical connector configured to detachably connect with and provide differential pressure data to patient monitoring equipment.

As illustrated in FIG. 1 showing patient monitoring system 1000, patient monitor cable 16 connects DPT system 10 to transport monitor 18. As noted above, patient monitoring system 1000 can be a transport environment in which transport monitor 18 is an mobile patient vitals monitor or similar device. More generally, however, transport monitor 18 is a first example of an electronic patient monitoring device, distinct from subsequent examples. Hereinafter transport monitor 18 is described as a device disposed to receive differential pressure data from DPT system 10 as analog electrical signals. These signals are generated by an onboard analog pressure sensor positioned proximate a fluid flowpath through DPT system 10, as described in detail with reference to later figures.

Patient monitoring system 2000 of FIG. 2 operates in substantially the same manner as patient monitoring system 1000 of FIG. 1, but includes separate primary and secondary monitors 22 and 24, as noted above. Primary monitor 22 and secondary monitor 24 are configured to receive different types of signals. To facilitate DPT system 10 serving both monitor types, DPT system 10 is configured to generate both types of signals, and multi-signal pressure cable 20 is configured to receive and split these two signal types into separate connector plugs each disposed to interface with either primary monitor 22 or secondary monitor 24. In short, and in a more general case, multi-signal pressure cable 20 has one input attachment and multiple output attachments. The single input attachment receives multiple signal types, while each output attachment provides only one of these signal types to an attached device. The remainder of this disclosure describes these different signal types principally as analog and digital electrical signals, but in the most general case other combinations of dissimilar signals may be benefit from the same treatment, e.g. digital electrical versus digital optical signals. Similarly, although this disclosure focuses on examples of DPT system 10 that are configured to produce two types of signals (i.e. analog and digital), alternatives with three or more dedicated sensors, or with any number of sensors producing three or more distinct signals, also fall within the scope and spirit of this disclosure.

As illustrated in FIG. 2, patient monitoring system 2000 includes patient monitor cable 16 for connection to secondary monitor 24. DPT system 10 generates digital and analog signals from separate pressure sensors within a shared housing, and provides these separate signals to primary and secondary monitors 22 and 24, respectively. Patient monitor cable 16 connects an analog output of multi-signal pressure cable 20 to secondary monitor 24, while the digital output of multi-signal pressure cable 20 is directly connected to primary monitor 22, which is configured to receive digital signals. FIGS. 1 and 2 depict the same cable or type of patient monitor cable 16 used to connect multi-signal pressure cable 20 to both (analog) transport monitor 18 and (analog) secondary monitor 24. In some cases, however, different or additional connectors can be included between multi-signal pressure cable 20 and either or both of primary and secondary monitors 22 and 24.

Although FIG. 2 illustrates both primary and secondary monitors as communicatively coupled to DPT system 10, either monitor may sometimes be redundant, depending on the specific environment of patient monitoring system 2000. In such cases, the multiple signal outputs of DPT system 10 and the forking arrangement of multi-signal pressure cable 20 permit a single DPT system to be used with whichever type of system is available or appropriate. In this way, DPT system 10 can serve as a versatile pressure sensing system regardless of monitor type, obviating any need to swap out one DPT system for another when, for example, transferring from an analog environment (e.g. with patient monitoring system 1000) to a digital one using primary monitor 22 (patent monitoring system 2000). The form and function of DPT system 10 are described in detail below.

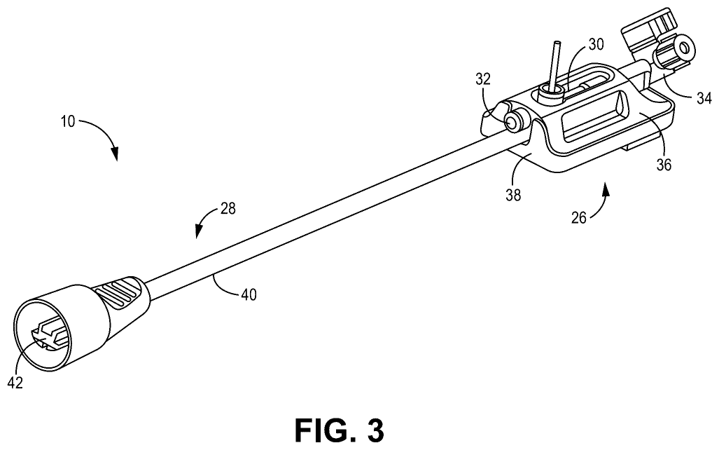

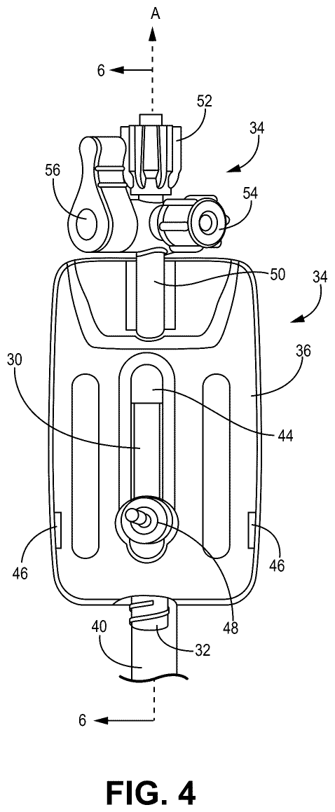

FIGS. 3 and 4 both illustrate DPT system 10 in greater detail. FIG. 3 provides a perspective view of DPT system 10, illustrating DPT sub-assembly 26 and hybrid connector 28. DPT sub-assembly 26 includes flowpath element 30 (with upstream attachment 32), stopcock 34, front cover 36, and rear cover 38. Hybrid connector 28 includes connector cable 40 and signal connector 42. FIG. 4 is a front plan view of the DPT system of FIG. 3, focusing on DPT sub-assembly 26.

DPT sub-assembly 26 conveys IV fluid and generates corresponding digital and analog differential pressure signals. Hybrid connector 28 conveys both sets of signals to a connected device configured to receive the digital or analog signals, or to a device (such as multi-signal pressure cable 20) capable of receiving both. In some examples, DPT system 10 may be a factory-sterilized, single-use kit. In the most general case, however, at least DPT sub-assembly 26 is sterilized prior to use.

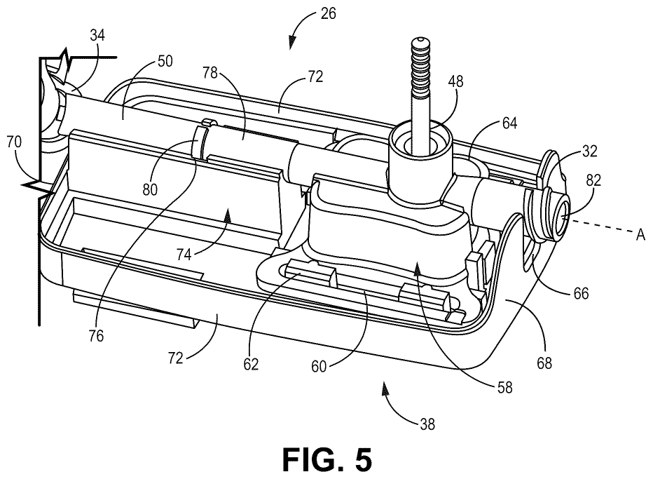

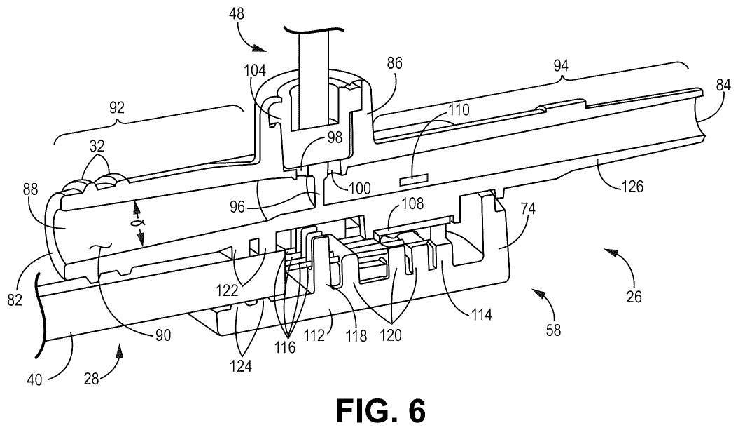

DPT sub-assembly 26 is a multi-sensor fluid handling device configured to receive IV fluid from fluid source 12 (see FIGS. 1 and 2), and deliver that IV fluid to a patient IV, e.g. via a needle or shunt, through stopcock 34. Flowpath element 30 of DPT sub-assembly 26 is a rigid body defining a fluid channel through DPT sub-assembly 26 (fluid channel 88; see FIGS. 5-6 below). Sensors within DPT sub-assembly 26 (e.g. sensor 108; see FIG. 6 below) are disposed adjacent to one another and in fluid communication with the interior of flowpath element 30 to sense differential pressure therein.

An upstream end of flowpath element 30 includes upstream attachment 32 to form a fluidically sealed connection with a fluid line from fluid source 12. In the example depicted in FIG. 3, upstream attachment 32 is a threaded front of flowpath element 30. In some cases, however, upstream attachment 32 can include other connecting or sealing features such as clamps or gaskets.

A downstream end of flowpath element 30 terminates at stopcock 34. Stopcock 34 is disposed downstream of sensor elements within DPT sub-assembly 26, and is a valve or valve capable of halting fluid egress from DPT sub-assembly 26.

Flowpath element 30 and other components of DPT sub-assembly 26 are enclosed between front cover 36 and rear cover 38. Front and rear covers 36 and 38 cooperate to form necessary fluid seals, secure connector cable 40, and support sensor elements as described in greater detail below with reference to FIG. 6. Front and rear covers 36 and 38 also define the form factor of DPT sub-assembly 26 into which most other components, including multiple sensors, fit.

FIG. 4 illustrates flowpath element 30, upstream attachment 32, and connector cable 40 of hybrid connector 28 as described above, and presents further details of stopcock 34, front cover 36, and rear cover 38. More specifically, FIG. 4 illustrates flowpath window 44 and cover snap attachments 46 of front cover 36, as well as flush tab 48 and flowpath connector 50, fluid line connectors 52 and 54, and stopcock lever 56 of stopcock 34. FIG. 4 also defines section plane 6-6, which provides the cross-section for FIG. 6 (discussed below).

Flowpath window 44 visually exposes flowpath element 30, allowing fluid flow through flowpath element 30 to be visible. Flowpath window 44 can, in some examples, be an aperture in front cover 36. In other examples, where such an aperture would interrupt the fluid seal provided by front and rear covers 36 and 38, respectively, about sensitive elements such as sensors and conductors contained therein, flowpath window 44 can be a transparent section of front cover 36 adjacent flowpath element 30. Cover snap attachments 46 secure front cover 36 snugly against rear cover 38, and can promote the aforementioned fluid seal. In some cases cover snap attachment 46 can consist of daggers or flanges extending from rear cover 38 and latching onto corresponding flanges or slots of front cover 36, as shown in FIG. 4. More generally, however, cover snap attachment 46 can include daggers or flanges extending from front cover 36 to rear cover 38, or both, in combination with other locking or latching mechanisms.

Flush tab 48, also called a snap tab, serves as a stop valve within flowpath element 30 preventing fluid flow from upstream attachment 32 to stopcock 34. In one example, flush tab 48 is a single-use flow blocking element installed in a state preventing fluid flow through flowpath element 30, as described below. Flush tab 48 prevents premature fluid flow through flowpath element 30, and is pulled outward (i.e. away from front cover 36 of DPT sub-assembly 26) to start flow through DPT sub-assembly 26.

As noted above, stopcock 34 is a fluid sealing valve disposed at a downstream end of flowpath element 30. Stopcock 34 is attached to flowpath element 30 via flowpath connector 50. As illustrated in FIG. 4, stopcock 34 can more specifically be a three-way valve with two fluid line connectors 52 and 54 configured to attach to downstream tubes or other fluid lines, e.g. to a patient needle or shunt, and/or to a drain. In the illustrated example, stopcock lever 56 is actuatable between at least three valve states: one connecting flowpath element 30 to fluid line connector 52, one connecting flowpath element 30 to fluid line connector 54, and one connecting fluid lines connectors 52 and 54 to each other. In some examples, stopcock lever 56 can also be actuated into a position fluidly isolating flowpath element 30 and both fluid line connectors 54 and 56 from each other.

FIGS. 5 and 6 provide views into the interior structure and fluid handling of DPT sub-assembly 26. FIG. 5 is a perspective view of DPT sub-assembly 26 with front cover 36 removed and depicts flowpath element 30 (with upstream attachment 32), stopcock 34, rear cover 38, and flush tab 48 generally as described above with reference to earlier figures. In addition, FIG. 5 illustrates structural housing 58, which includes snap slot 60 for snap attachment 62 to rear cover 38, and flowpath recess 64 to receive flowpath element 30. FIG. 5 also illustrates sections of rear cover 38 not visible in previous figures, including cover opening 66 through cover inlet side 68, cover outlet and lateral sides 70 and 72, respectively, and pedestal 74 with retaining slot 76. Flowpath connector 50 includes flowpath connector sleeve 78 with collar 80 received within retaining slot 76. Flowpath inlet 82 of flowpath element 30 is also visible in FIG. 5.

Structural housing 58 is a substantially rigid support body disposed between front and rear covers 36 and 38, respectively. Structural housing 58 supports and retains flowpath element 30 in a fixed position relative to both covers, and relative to stopcock 34. In the example shown, structural housing 58 features symmetrically disposed snap slots 60 (only one is visible in FIG. 5; the remaining snap slot is located on the opposite side of structural housing 58, behind flowpath element 30) through which snap attachments 62 latch to lock structural housing 58 into place. Snap attachments 62 can, as shown, be barbed flanges extending from rear case 38. Structural housing 58 also includes flowpath recess 64, a fitted bowl or partial enclosure that receives and positions flowpath element 30 relative both to rear cover 38 and to sensitive electronics (see FIG. 6).

As described previously, rear cover 38 cooperates with front cover 36 to enclose flowpath element 30 and structural housing 58. As shown in FIG. 5, rear cover 38 includes cover opening 66 allowing upstream attachment 32 to pass through cover inlet side 68, while the opposite cover outlet side 70 does not obstruct stopcock 34, which is instead surrounded by front cover 36. Cover lateral sides 72 are flanges on opposite sides of rear cover 38 that, together with cover inlet and outlet sides 68 and 70, respectively, define a perimeter flange that closely mates with front cover 36.

Rear cover 38 also includes pedestal 74, a structural support that abuts flowpath connector sleeve 78, an upstream-most portion of stopcock 34 that surrounds the downstream-most portion of flowpath element 30. Pedestal 74 includes retaining slot 76, a hemi-cylindrical groove aligned with collar 80, a corresponding alignment and retention feature of flowpath connector sleeve 78. During installation, flowpath connector sleeve 78 is fitted about the downstream end of flowpath element 30, then pressed down into pedestal 74 such that collar 80 is retained axially by retaining slot 76. This attachment both supports flowpath element 30 and stopcock 34 and prevents stopcock 34 from disengaging from flowpath 30.

FIG. 6 depicts internals of DPT sub-assembly 26 and connected components as described above with respect to FIG. 5, excluding stopcock 34 and rear cover 38. FIG. 6 is a perspective cross-sectional view along section plane 6-6 of FIG. 4, and illustrates hybrid connector 28 (with connector cable 40), flowpath element 30, flush tab 48, structural housing 58, and flowpath inlet 82 as described above. Additionally, FIG. 6 illustrates a variety of fluid flow-related elements, including flowpath outlet 84, flush tab collar 86, fluid channel 88 defined by channel wall 90 and separated into upstream channel section 92 and downstream channel section 94 by channel dividing wall 96, flush section inlet and outlet 98 and 100, respectively, flush section chamber 102, and flush tab plunger 104 with flush tab pull 106. FIG. 6 also introduces sensor 108, which is fluidly connected to fluid channel 88 by sensor cutout 110. In addition to pedestal 74, discussed previously, structural housing 58 also includes structural housing base 112 and sensor supports 114. Connector cable 40 contains multiple conductors 116 separated and aligned by conductor separators 118 and positioned by conductor supports 120. Clamping flange 122 of flowpath element 30 and clamping flange 124 of structural housing 58 both abut connector cable 40, and flowpath element 30 includes stopcock abutting section 126 near flowpath outlet 84.

Flowpath element 30 extends along axis A and includes flush tab collar 86 surrounding and retaining flush tab 48. Flowpath element 30 defines fluid channel 88, an approximately cylindrical passage circumferentially defined by channel wall 90 and oriented along axis A. Flowpath element 30 extends from an upstream end at flowpath inlet 82 adjacent upstream attachment 32 to a downstream end at flowpath outlet 84 adjacent stopcock 34. As illustrated in FIG. 6, fluid channel 88 has tapering and flaring diameter that is narrowest (with diameter d) where interrupted by channel dividing wall 96. Diameter d can, for example, be 1 to 8 mm. Channel dividing wall 96 is axially aligned with flush tab 48 and divides fluid channel 88 into upstream channel section 92 and downstream channel section 94. Upstream channel section 92 extends from flowpath inlet 82 to channel dividing wall 96, and downstream channel section 94 extends from channel dividing wall 96 to flowpath outlet 84. Upstream and downstream channel sections 92 and 94 are connected solely through flush tab 48. More specifically, flush tab 48 includes flush section inlet 98 and flush section outlet 100 which provide flow ingress to and egress from flush section chamber 102, respectively from upstream channel section 92 and to downstream channel section 94. Flush tab plunger 104, a rubberized or other malleable stopper, defines flush section chamber based on its position relative to flush section inlet and outlet 98 and 100, respectively. Flush tab pull 106 provides a handle to pull flush tab plunger 104 away from flush section inlet 98 and flush tab outlet 100. When in a closed position, flush tab plunger 104 prevents fluid flow through flowpath element 30 by blocking flush section inlet 98 and flush section outlet 100. When withdrawn radially away from axis A of flowpath element 30, flush section chamber 102 fluidly connects upstream channel section 92 to downstream channel section 94. As discussed above, some examples of flush tab 48 can be single-use seals not designed to be re-closed once opened, i.e. once flush tab plunger is withdrawn radially away from flowpath element 30.

FIG. 6 also illustrates sensor 108, a differential pressure transducer disposed alongside and abutting flowpath element 30. Although only one sensor 108 is shown in FIG. 6, a second sensor 108 can be located forward of the section plane. Sensor cutout 110 provides a fluid connection between fluid channel 88 and sensor 108. As illustrated in FIG. 6, sensor cutout 110 is axially elongated, and is situated within downstream channel section 94.

Structural housing 58 includes structural housing base 112, a substantially flat plate anchored to rear cover 38 (see FIG. 5). Multiple flanges or fingers extend upward from structural housing base 112, including sensor support 114, conductor separator 118, conductor supports 120, and clamping flange 124. Sensor support 114 act as a pedestal retaining sensor 108 against flowpath element 30. Sensor 108 generates sensor data in the form of sensor signals transmitted through connected conductors 116. In the illustrated example, four separate conductors 116 are attached to separate contacts of sensor 108. Where conductors 116 leave connector cable 40, they are separated into a common plane by conductor separator 118. Conductor separator 118 is a flange extending vertically from structural housing base 112 to support conductors 116, with fingers extending between each conductor 116 and adjacent conductors. Conductors 116 are also supported and aligned by conductor supports 120 between conductor separator 118 and contacts of sensor 108.

Connector cable 40 surrounds conductors 116 in an insulating and protective cover that is clamped between clamping flanges 122, which extend toward structural housing base 112 from flowpath element 30, and clamping flanges 124, which extend upward from structural housing base 112 toward flowpath element 30. This clamping both retains connector cable 40 to reduce or eliminate tension on conductors 116 that could break contacts with sensor 108, and forms a fluid seal about connector cable to protect sensor 108 and conductors 116 from shorts.

Flowpath element 30 terminates axially at flowpath abutting section 126, a tapering section disposed radially inward of and snugly overlapping flowpath connector sleeve 78 (see FIG. 5).

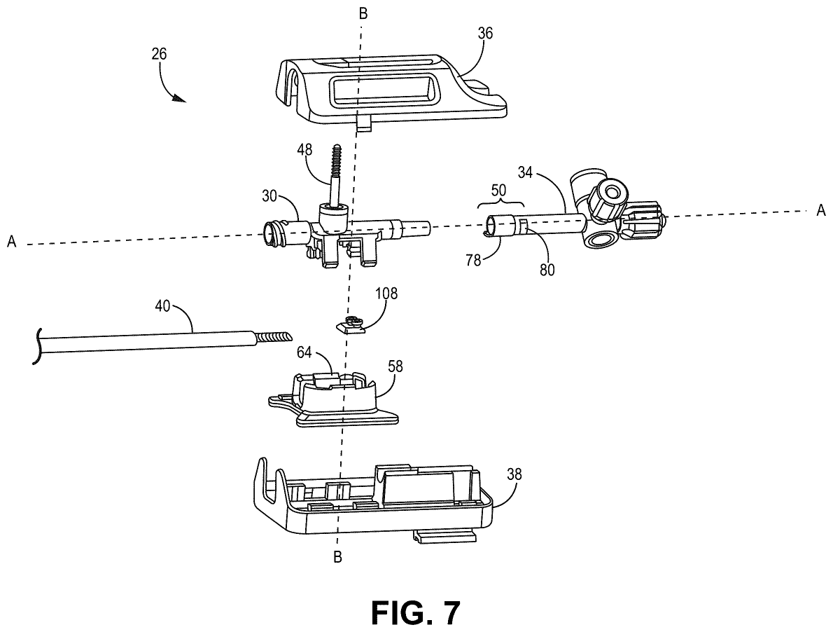

FIG. 7 is an exploded view of components of the DPT sub-assembly 26 and assembly features of DPT sub-assembly 26. Front cover 36 and rear cover 38 have been displaced along axis B-B to reveal flowpath element 30, stopcock 34, flowpath connector 50, structural housing 58, and sensors 108. Flowpath element 30 has been displaced from structural housing 58 along axis B-B to reveal connector cable 40 and sensors 108. Stopcock 34 and flowpath connector 50, which includes flowpath connector sleeve 78 and collar 80, have been separated from flowpath element 30 along axis A-A. As depicted, flush tab 48 is installed within flowpath element 30.

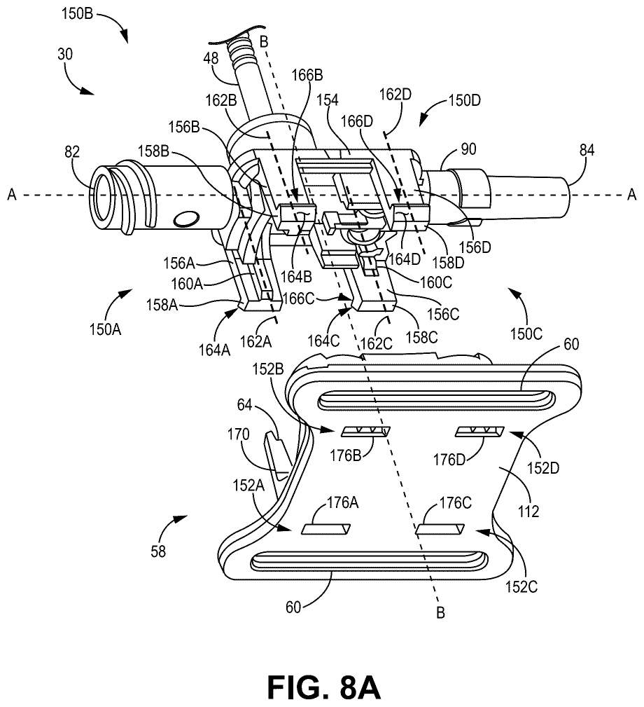

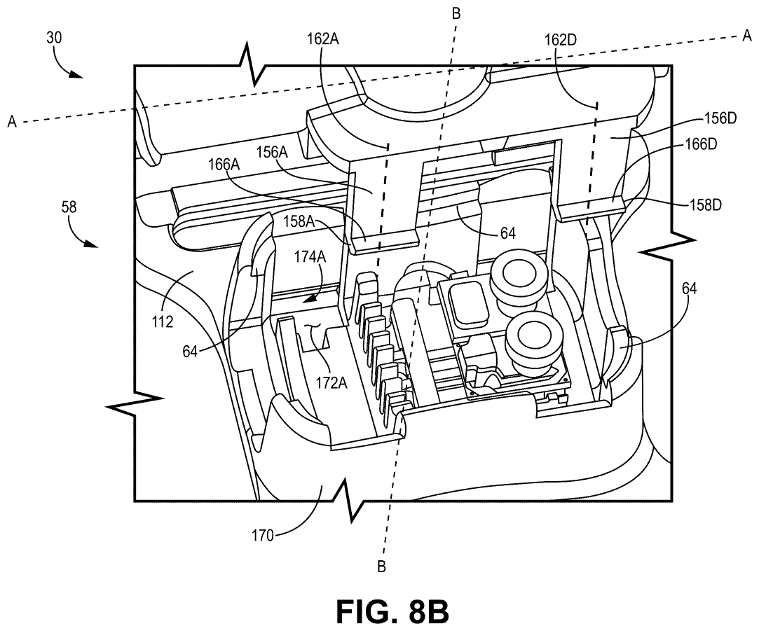

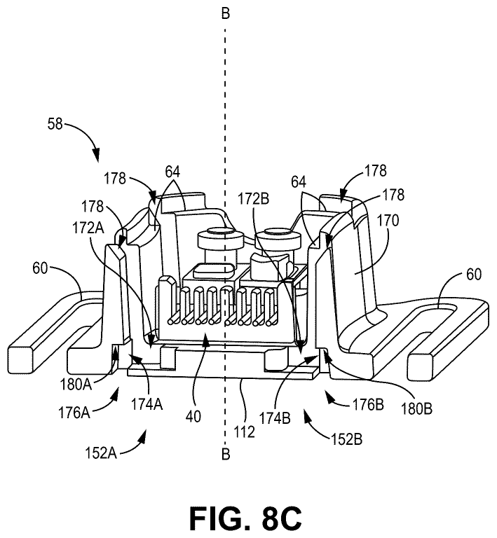

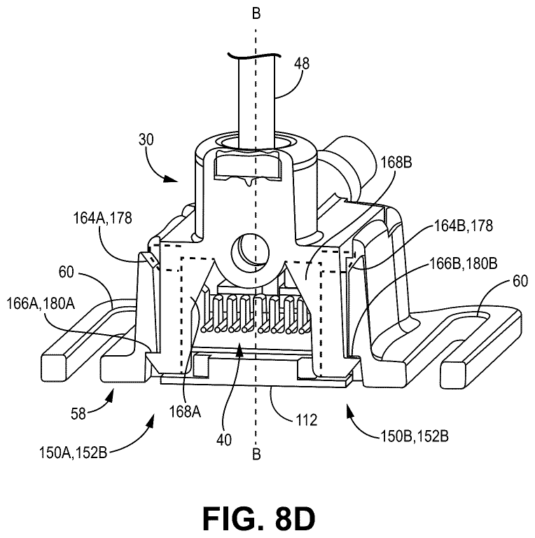

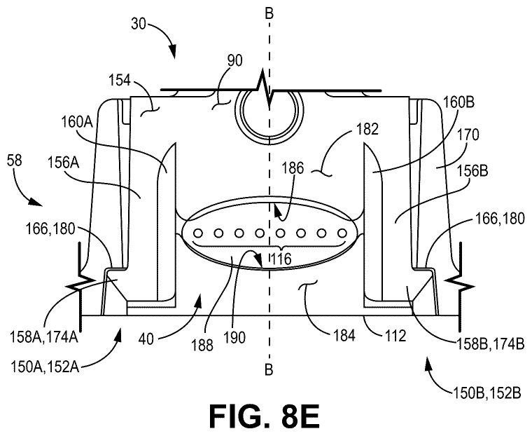

DPT sub-assembly 26 includes several features that simplify assembly, eliminate manufacturing steps, and/or reduce manufacturing time. For instance, a snap fit can secure flowpath element 30 to structural housing 58. In the example depicted by FIGS. 7, 8A, 8B, 8C, and 8D, the snap fit includes protrusions 150A, 150B, 150C, and 150D extending from flowpath element 30 that are received by receptacles 152A, 152B, 152C, and 152D of structural housing 58. In other examples, protrusions 150A, 150B, 150C, and 150D may extend from structural housing 58 to engage receptacles 152A, 152B, 152C, and 152D of flowpath element 30. During assembly, flowpath element 30 translates along axis B-B until protrusions 150A, 150B, 150C, and 150D engage receptacles 152A, 152B, 152C, and 152D and flowpath recess 64 receives an outer periphery of flowpath element 30 to secure flowpath element 30 to structural housing 58.

In each of the examples, protrusions 150A, 150B, 150C, and 150D are parallel to each other along axis B-B facilitating engagement with receptacles 152A, 152B, 152C and 152D along a single line of action (i.e., axis B-B). Processes completed with a minimum number of actions simplify the assembly process relative to processes requiring multiple steps assembled along multiple lines of action. In this instance, the assembly of flowpath element 30 to structural housing 58 requires applying force to flowpath element 30 along axis B-B only, thereby simplifying the assembly of DPT sub-assembly 26. Further improvement to the assembly process is provided through automated assembly techniques, which are more easily implemented when assembly steps and lines of action are minimized.

FIG. 8A and FIG. 8B are perspective views of flowpath element 30 and structural housing 58 prior to assembly that illustrate protrusions 150A, 150B, 150C, and 150D as well as receptacles 152A, 152B, 152C, and 152D of an exemplary snap fit. While the present example depicts four protrusions 150A, 150B, 150C, and 150D and four receptacles 152A, 152B, 152C, and 152D, the snap fit may be formed by fewer or more protrusions with an equal number of receptacles in other examples.

As shown, flowpath element 30 includes lateral plate 154 that extends transversely away from channel wall 90 such that axis B-B is normal to lateral plate 154. Protrusions 150A, 150B, 150C, and 150D include respective beams 156A, 156B, 156C, and 156D; barbs 158A, 158B, 158C, and 158D; and in some examples, ribs 160A, 160B, 160C, and 160D. Beams 156A, 156B, 156C, and 156D extend perpendicularly from exterior corners of lateral plate 154 parallel to axis B-B. The extension direction of each beam is defined by respective beam axes 162A, 162B, 162C, and 162D. Each beam axis extends longitudinally through a geometric center of respective beam cross-sections. Distal ends of beams 156A, 156B, 156C, and 156D opposite lateral plate 154 include barbs 158A, 158B, 158C, and 158D, which protrude laterally outward from respective beams 156A, 156B, 156C, and 156D. Each barb 158A, 158B, 158C, and 158D defines a truncated triangular section when viewed along axis A-A formed by respective tapered surfaces 164A, 164B, 164C, and 164D and respective lip surfaces 166A, 166B, 166C, and 166D. Tapered surfaces 164A, 164B, 164C, and 164D face away from lateral plate 154 such that tapered surfaces 164A, 164B, 164C, and 164D engage mating surfaces of structural housing 58 during insertion.

In some examples, one or more beams 156A, 156B, 156C, and 156D are equipped with rib 160A, 160B, 160C, or 160D to increase flexural modulus of respective protrusions 150A, 150B, 150C, and 150D. Each of ribs 160A, 160B, 160C, and 160D extends longitudinally along one of beams 156A, 156B, 156C, and 156D from lateral plate 154 up to respective barbs 158A, 158B, 158C, and 158D, or an intermediate distance between lateral plate 154 and respective barbs 158A, 158B, 158C, and 158D. The cross-section normal to beam axes 162A, 162B, 162C, and 162D and length of each rib 160A, 160B, 160C, and 160D can be tailored for each beam 156A, 156B, 156C, and 156D, some protrusions 150A, 150B, 150C, and 150D having greater or lesser flexural modulus than other protrusions 150A, 150B, 150C, and 150D. Ribs 160A, 160B, 160C, and 160D can have constant or variable cross-sectional areas along beam axes 162A 162B, 162C, and 162D, and in some examples, form gussets 168A, 168B, 168C, and 168D (see FIG. 8D) joining beams 156A, 156B, 156C, and 156D to lateral plate 154 to further increase the flexural moduli of one or more protrusions 150A, 150B, 150C, and 150D.

As shown in FIG. 8A, upstream beams 156A and 156B are closest to flowpath inlet 82 while downstream beams 156C and 156D are closest to flowpath outlet 84. Upstream beams 156A and 156B include ribs 160A and 160B, which have larger cross-sectional areas than corresponding ribs 160C and 160D of downstream beams 156C and 156D. Ribs 160A and 160B extend an entire length of beams 156A and 156B from lateral plate 154, while ribs 160C and 160D extend an intermediate distance from lateral plate 154 toward barbs 158C and 158D as represented by rib 160C depicted in FIG. 8A. Further, a cross-sectional area of ribs 160A and 160B increases towards lateral plate 154 to form gussets 168A and 168B (see FIG. 8D). In the depicted example, upstream beams 156A and 156B and associated ribs 160A and 160B are identical. Similarly, downstream beams 156C and 156D and associated ribs 160C and 160D are identical. Accordingly, the flexural moduli about a minor dimension of beams 156A and 156B have been increased to a greater degree than the flexural moduli about the minor dimension of beams 156C and 156D. In other examples, flexural moduli about the minor dimension of beams 156C and 156D may be greater than beams 156A and 156B. In still other examples, each of beams 156A, 156B, 156C, and 156D may have a different flexural modulus.

Increased flexural moduli about the minor dimension of beams 156 increases the insertion force as well as the extraction force necessary to separate flowpath element 30 from structural housing 58, the increase to insertion force not necessarily equal to the increase to extraction force. Geometry of protrusions 150A, 150B, 150C, and 150D, such as the length, width, and thickness of beams 156A, 156B, 156C, and 156D; and/or ribs 160A, 160B, 160C, and 160D, as well as material of flowpath element 30 can be selected to tailor the insertion force and/or extraction force. In some examples the force required to engage protrusions 150A, 150B, 150C, and 150D of flowpath element 30 with receptacles 152A, 152B, 152C, and 152D of structural housing 58 ranges between four kilograms to fourteen kilograms (or about nine pounds to thirty pounds). Extraction force of the same example may range between nine kilograms to sixteen kilograms (or about twenty pounds to thirty-five pounds). In some examples, extraction force exceeds eleven kilograms (or about twenty-five pounds).

Each of receptacles 152A, 152B, 152C, and 152D is formed by a void, cavity, or slot formed in structural housing base 112 and/or side walls 170 of structural housing 58. Receptacles 152A, 152B, 152C, and 152D include respective pockets 172A, 172B, 172C, and 172D; undercuts 174A, 174B, 174C, and 174D; and in some examples openings 176A, 176B, 176C, and 176D. Pockets 172A, 172B, 172C, and 172D are open along axis B-B to receive barbs 158A, 158B, 158C, and 158D of protrusions 150A, 150B, 150C, and 150D. Cross-sectional areas of pockets 172A, 172B, 172C, and 172D normal to axis B-B accommodate protrusions 150A, 150B, 150C, and 150D throughout the insertion of flowpath element 30 into structural housing 58. For example, cross sectional areas of one or more pockets 152A, 152B, 152C, and 152D can be extended in a direction of deflection associated with each of protrusions 150A, 150B, 150C, and 150D during insertion. Referring to the exampled depicted by FIGS. 8A and 8B, beams 156A and 156C bend inward towards opposing beams 156B and 156D, which also deflect inward during insertion. Pockets 172A, 172B, 172C, and 172D can extend further inward (i.e., towards axis A-A) to accommodate respective beams 156A, 156B, 156C, and 156D (i.e., a larger lateral dimension) while a center portion of pockets 172A, 172B, 172C, and 172D can be extended, if necessary, to accommodate ribs 160A, 160B, 160C, and 160D as shown in FIG. 8A. Accordingly, cross-sectional areas of pockets 172A, 172B, 172C, and 172D normal to axis B-B conform to cross-sections of respective beams 156A, 156B, 156C, and 156D and, where necessary for insertion, cross-sections of respective ribs 162A, 162B, 162C, and 162D.

Undercuts 174A, 174B, 174C, and 174D extend into structural housing 58 from one of pockets 172, 172B, 172C, and 172D and represent portions of receptacles 152A, 152B, 152C, and 152D not open to flowpath element 30 along axis B-B. Undercuts 174A, 174B, 174C, and 174D receive one of barbs 158A, 158B, 158C, and 158D after full insertion of flowpath element 30 into structural housing 58. FIG. 8A also depicts openings 176A, 176B, 176C, and 176D, an optional feature of receptacles 156A, 156B, 156C, and 156D. Openings 176A, 176B, 176C, and 176D extend through structural housing base 112 opposite pockets 172A, 172B, 172C, and 172D to intercept one of undercuts 174A, 174B, 174C, and 174D. Openings 176A, 176B, 176C, and 176D permit formation of undercuts 174A, 174B, 174C, and 174D using a two-part molding process in which a first mold forms pockets 172A, 172B, 172C, and 172D and a second mold forms openings 176A, 176B, 176C, and 176D and undercuts 174A, 174B, 174C, and 174D as well as other features of structural housing 58. Forming structural housing 58 using two-part molds as opposed to three-part molds (or more) further simplifies the manufacturing of DPT subassembly 26.

FIG. 8C is a perspective cross-sectional view that is normal to axis A-A depicting receptacles 152A and 152B of structural housing 58. Features of receptacles 152A and 152B are representative of analogous features of receptacles 152C and 152D (not shown in FIG. 8C and FIG. 8D). While the size and shape of receptacles 152C and 152D may differ from receptacles 152A and 152B, the features of receptacles 152A and 152B function in the same manner as analogous features of receptacles 152C and 152D.

As shown by the depicted example, pockets 172A and 172B extend into structural housing base 112 along axis B-B from a first side while openings 176A and 176B extend into structural housing base 112 from an opposite second side. Undercuts 174A and 174B extend laterally outward from respective pockets 172A and 172B under side wall 170 of structural housing 58. Center portions of pockets 172A and 172B extend laterally inward to a greater extent than peripheral portions of pockets 172A and 172B to accommodate full-length ribs 160A and 160B of upstream protrusions 150A and 150B. Since protrusions 150C and 150D do not include full-length ribs, center portions of pockets 172C and 172D (not shown) are not necessarily extended laterally inward to a greater extent than peripheral portions of pockets 172C and 172D.

FIG. 8C depicts additional features of structural housing 58 that aid assembly with flowpath element 30. Structural housing 58 includes flowpath recess 64 defined about a distal periphery of side wall 170. Flow path recess 64 defines a lip conforming to a periphery of lateral plate 154 and a ledge abutting lateral plate 154 to support flowpath element 30. In some examples, structural housing 58 includes shelf faces 178 formed at the distal periphery of side wall 170 coinciding with each receptacle 152A, 152B, 152C, and 152D. Shelf faces 178 are oblique to axis B-B and complementary to respective tapered surfaces 164A, 164B, 164C, and 164D of flowpath element 30. As shown, shelf faces 178 are angled towards the interior of structural housing 58, while tapered surfaces 164A, 164B, 164C, and 164D are angled towards an exterior of flowpath element 30.

FIG. 8D is a cross-sectional view through flowpath element 30, structural housing 58, and connector cable 40 parallel to axis B-B depicting complementary geometry of beams 156A and 156B; barbs 158A and 158B; pockets 172A and 172B; and undercuts 174A and 174B. While protrusion 150A, protrusion 150B, receptacle 152A, and receptacle 152B illustrate features of the snap fit connection between flowpath element 30 and structural housing 58, analogous features of protrusion 150C, protrusion 150D, receptacle 152C, and receptacle 152D function in the same manner described. Initially, tapered surfaces 164A, 164B, 164C, and 164D abut shelf faces 178 during insertion of flowpath element 30 into structural housing 58 as depicted by dashed lines in FIG. 8D, which are representative of tapered faces 164A and 164B. Applying an insertion force to flowpath element 30 along axis B-B causes beams 156A, 156B, 156C, and 156D (beams 156C and 156D not shown) to deflect inward to clear shelf faces 178. Once engaged to structural housing 58, lip surfaces 166A, 166B, 166C, and 166D of barbs 158A, 158B, 158C, and 158D abut restraining surfaces 180A, 180B, 180C, and 180D of respective undercuts 174A, 174B, 174C, and 174D to restrain flowpath element 30 relative to structural housing 58 along axis B-B (undercuts 174C and 174D not shown).

FIG. 8E is a cross-sectional view normal to axis A-A taken through flowpath element 30, structural housing 58, and connector cable 40 depicting another feature of the flowpath snap fit. Flowpath element 30 includes cable pedestal 182, and structural housing 58 includes saddle 184. Cable pedestal 182 is a beam member extending from channel wall 90 and/or lateral plate 154 towards connector cable 40. In some examples, cable pedestal 182 is centrally located between two of protrusions 150A, 150B, 150C, and 150D. As shown, cable pedestal 182 extends between upstream protrusions 150A and 150B. Terminal face 186 of cable pedestal 182 engages protective cover 188 of connector cable 40. Terminal face 186 can be flat or profiled to accommodate protective cover 188 of connector cable 40.

Saddle 184 is a region of structural housing 58 that engages protective cover 188 of connector cable 40 opposite cable pedestal 182. In some examples, saddle 184 is a recessed region of structural housing base 112. In other examples, saddle 184 may protrude from structural housing base 112 to engage protective cover 188 of connector cable 40 at saddle surface 190. Like terminal face 186, saddle surface 190 can be flat or profiled to accommodate protective cover 188 of connector 40. Exemplary profiles for terminal face 186 and saddle surface 190 include semicylindrical, semielliptical, semicircular, or semiovular. Terminal face 186 and saddle surface 190 profiles may be the same or different. As shown in FIG. 8E, terminal face 186 and saddle surface 190 are semielliptical, each engaging a semielliptical exterior profile of connector cable 40.

Protective cover 188 of connector cable 40 provides electrical insulation and environmental protection for conductors extending between sensors 108 and signal connector 42. Additionally, protective cover 188 of connector cable 40 is a resilient material that exerts a restoring force under compression and, accordingly, functions as a spring. One or both of cable pedestal 182 and saddle 184 may interfere with protective cover 188 of connector cable 40 when flowpath element 30 is fully inserted into structural housing 58. For example, the interference between protective cover 188 and cable pedestal 182 as well as the interference between protective cover 188 and saddle 184 can be from about 0.10 millimeters to about 0.50 millimeters inclusive (i.e., approximately from 0.004 inches to 0.020 inches inclusive). In some examples, the interference between protective cover 188 and cable pedestal 182 is the same as the interference between protective cover 188 and saddle 184, while in other examples the interference of each interface can be different. The interference between protective cover 188 and one or both of cable pedestal 182 and saddle 184 imposes a restoring force onto flowpath element 30 along axis B-B, displacing lip surfaces 166A, 166B, 166C, and 166D of flowpath element barbs 158A, 158B, 158C, and 158D into engagement with restraining surfaces 180A, 180B, 180C, and 180D of structural housing undercuts 174A, 174B, 174C, and 174D.

Once lip surfaces 166A, 166B, 166C, and 166D abut respective restraining surfaces 180A, 180B, 180C, and 180D, certain examples can include residual interference between protective cover 188 and cable pedestal 182, saddle 184, or both. The residual interference compresses protective cover 188 and restrains movement of connector cable 40 along axis A-A. This restraint of connector cable 40 serves as a strain relief for conductors 116 that connect to sensors 108 within DPT subassembly 26. Any residual interference also serves to inhibit relative movement of flowpath element 30 and structural housing 58 along axis B-B, reducing or eliminating rattling of DPT subassembly 26. Furthermore, residual compressive stress can prevent damage to sensor 108 during the assembly process and subsequent use by driving flowpath element 30 and structural hosing 58 apart and driving lip surfaces 166A, 166B, 166C, and 166D of protrusions 150A, 150B, 150C, and 150D into engagement with retention surfaces 180A, 180B, 180C, and 180D of undercuts 174A, 174B, 174C, and 174D.

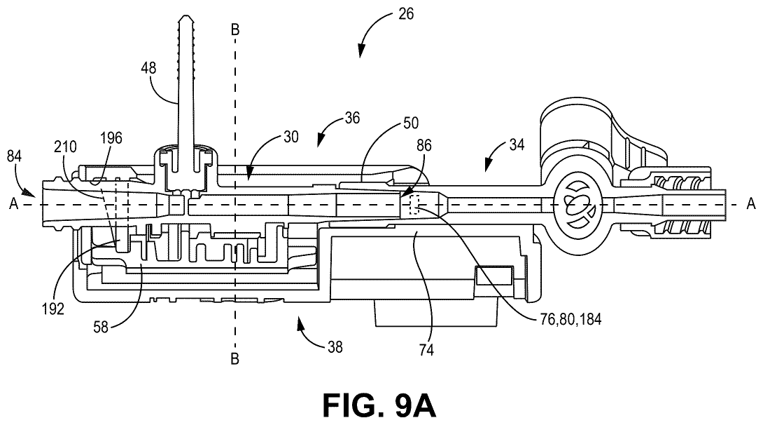

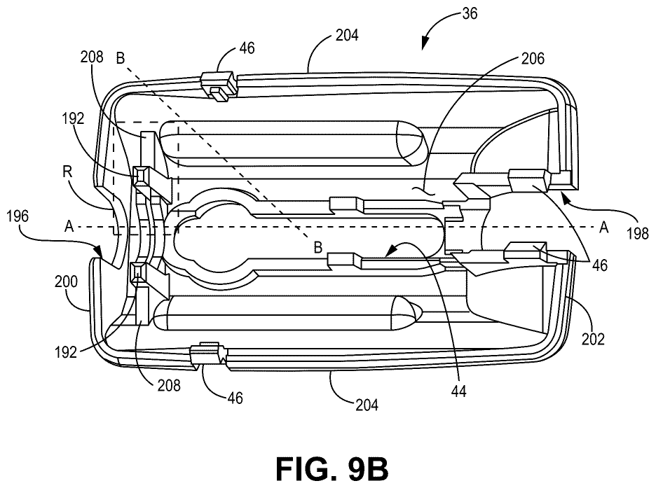

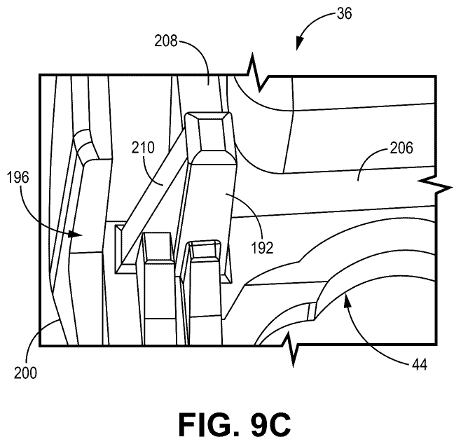

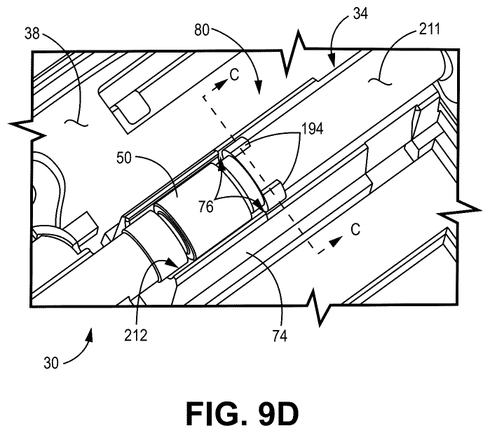

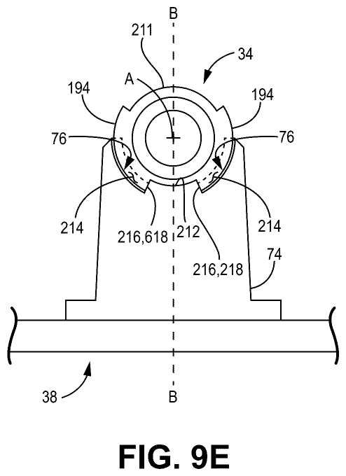

FIG. 9A is a cross-sectional view of an assembled DPT subassembly 26 taken along axis A-A perpendicular to axis B-B. FIG. 9A depicts another construction simplification in which flowpath element 30, stopcock 34, and structural housing 58 are restrained along axis A-A relative to front cover 36 and rear cover 38 without adhesive.

The restraint includes one or more cantilevered supports 192, flowpath connector 50, pedestal 74, and one or more segments 194 of collar 80. Cantilevered supports 192 are beam members that extend from front cover 36 or rear cover 38 to elastically engage structural housing 58 to apply a restraining force along axis A-A towards stopcock 34. Flowpath connector 50 forms an integral part of or is affixed to stopcock 34. An interior bore of flowpath connector 50 tapers from a maximum dimension at its extremity to a minimum dimension within stopcock 34. Adjacent to flowpath connector 50, collar segments 194 engage one or more retaining slots 76 of rear cover pedestal 74 to oppose the restraining force of cantilevered supports 192. Accordingly, the restraining force applied by one or more cantilevered supports 192 along axis A-A to structural housing 58 transfers to flowpath element 30 via flowpath recess 64 (not shown in FIG. 9A). Under action of the restraining force, flowpath element 30 engages a tapered bore of flowpath connector 50. One or more collar segments 194 of stopcock 34 are forced into engagement with retaining slot or slots 76 of pedestal 74 to oppose the restraining force and thereby inhibit displacement of structural housing 58, flowpath element 30, connector sleeve 50, and stopcock 34 along axis A-A.

FIG. 9B is a perspective view of an interior of front cover 36 that includes an exemplary implementation of cantilevered supports 192. Front cover 36 includes flowpath window 44, cover snap attachments 46, cover inlet opening 196, cover outlet opening 198, cover inlet side 200, cover outlet side 202, lateral sides 204, and front side 206. Lateral sides 204, cover inlet side 200, and cover outlet side 202 define a perimeter flange that closely mates with rear cover 38. Front side 206 spans between and joins cover inlet side 200, cover outlet side 202, and lateral sides 204. Cover snap attachments 46 extend from the perimeter flange to mate with corresponding features of rear cover 38 to join front cover 36 to rear cover 38. Cover inlet opening 196 penetrates cover inlet side 200 to provide clearance for flowpath element 30. Cover outlet opening 198 extends through cover outlet side 202 to provide clearance for flowpath element 30, flowpath connector 50, and stopcock 34. Flowpath window 44 extends through front side 204 to visually expose flowpath element 30 from an exterior of front cover 36.

Cantilevered supports 192 extend away from an interior of front cover 36 (i.e., from front side 206). In the depicted example, front cover 36 includes two cantilevered supports 192, each support 192 located on opposite sides of cover inlet opening 196. Cantilevered supports 192 are closer to and adjacent cover inlet side 200 such that engagement with structural housing 58 applies a restraining force along axis A-A towards cover outlet opening 198 and stopcock 34 as shown in FIG. 9A. Front cover 36 may include side gussets 208 that extend from a lateral side of each cantilevered support 192 towards lateral sides 204 and front side 206 to increase lateral stiffness of front cover 36. Cantilevered supports 192 may include longitudinal ribs 210 (see FIG. 9C) that extend along a side of each spring support 192 opposite structural housing 58 to resist deflection imposed on cantilevered supports 192 by engagement with structural housing 58.

FIG. 9C is a perspective view of region R of FIG. 9B as viewed from cover inlet side 200 that depicts cantilevered support 192 and longitudinal rib 210 in greater detail, which are also depicted with dashed lines in FIG. 9A. The restraining force applied by each cantilevered support 192 to structural housing 58 can be tailored by adjusting geometry of cantilevered supports 192. Factors affecting the flexural modulus of cantilevered supports 192 include a cross-sectional area/shape of each cantilevered support 192 taken normal to a longitudinal direction (i.e., extension direction) of respective cantilevered supports 192, a length of cantilevered supports 192 measured from front cover 36 to a distal tip thereof, and a longitudinal location of a zone of contact with structural housing 58. As depicted by FIG. 9A, cantilever supports 192 engage a surface of structural housing 58 that faces cover inlet opening 196.

In some examples, the flexural modulus of cantilevered supports 192 can be augmented with longitudinal ribs 210. Longitudinal ribs 210 extend along a side of each cantilevered support 192 that opposes engagement with structural housing 58. A cross-sectional area of each longitudinal rib 210 supplements a cross-sectional area of cantilevered supports 192. In each case, the cross-sectional area of cantilevered supports 192, longitudinal ribs 210, or both can vary along a longitudinal direction of one or more cantilevered supports 192. As shown, a cross-sectional area of cantilevered supports 192 remains constant along the longitudinal direction of the cantilevered supports 192 while the cross-sectional area of longitudinal ribs 210 varies from front cover 36 towards the distal tips (i.e., free ends) of cantilevered supports 192. Specifically, the cross-sectional area of longitudinal ribs 210 decreases linearly from front cover 36 towards the distal tips (i.e., free ends) of cantilevered supports 192. In other examples, the cross-sectional area of longitudinal ribs can include regions of constant cross-sectional area combined with regions of increasing or decreasing cross-sectional area.

FIG. 9D is a perspective detail view showing the interface of flowpath element 30, flowpath connector 50, and stopcock 34 with pedestal 74. Flowpath connector 50 receives flowpath element 30 and is demarked by a larger diameter region of stopcock 34. Adjacent to flowpath connector 50, stopcock 34 includes collar 80. Collar 80 may include one or more collar segments 194 that extend circumferentially about cylindrical body 211 of stopcock 34. Pedestal 74 includes semicylindrical channel 212 that extends along axis A-A and receives cylindrical portions of flowpath element 30, flowpath connector 50, and stopcock 34. At least one retaining slot 76 extends into pedestal 74 from semicylindrical channel 212 and receives an equal number of collar segments 194.

FIG. 9E is a cross-sectional view taken along line C-C in FIG. 9D, depicting stopcock 34, collar segments 194, pedestal 74, and retaining slots 76. In the depicted example, stopcock 34 includes two collar segments 194 each extending circumferentially about cylindrical body 211 to subtend a sector of stopcock 34. Collar segments 194 are semiannular and are circumferentially spaced from each other. As depicted, collar segments 194 are symmetrical about a plane bisecting pedestal 74 and intersecting axis A-A. Pedestal 74 includes two retaining slots 76, each retaining slot 76 receiving one of the collar segments 194 of stopcock 34. Accordingly, each retaining slot 76 subtends a sector of semicylindrical channel 212 that coincides with one of collar segments 194. Retaining slots 76 include end faces 214 that abut side faces of collar segments 194 to oppose the restraining force produced by cantilevered supports 192. Additionally, circumferential faces 216 of retaining slots 76 abut corresponding circumferential faces 218 of collar segments 194 to restrain stopcock 34 against rotation about axis A-A.

Referring to FIG. 5 and FIG. 9A, a partial DPT sub-assembly 26 consisting of stopcock 34, flowpath element 30, and structural housing 50 engages rear cover 38 at snap attachments 62 and retaining slots 76 of pedestal 74 while engaging front cover 36 at cantilevered supports 192. Partial DPT sub-assembly 26 is retained to rear cover 38 along the axis B-B by snap attachments 62 engaging snap slots 60 of structural housing 58. Cantilevered supports 192 elastically bend, deflecting in a direction parallel to axis A-A. The elastic engagement of cantilevered supports 192 to structural housing 58 applies a retaining force to structural housing 58 along axis A-A. Retaining slot 76 receives collar 80, which may include one or more segments 194, to react retaining force. A distance measured parallel to axis A-A between outward facing surfaces of collar segments 194 and surfaces of structural housing 58 abutting cantilevered supports 192 is greater than a distance between interior-facing end faces 214 of retaining slots 76 and contact surfaces of cantilevered supports 192. Interference between structural housing 58 and cantilevered supports 192 elastically bend cantilevered supports 192 in an outward direction along axis A-A. Cantilevered supports 192, collar 80 (i.e., collar segments 194), and retaining slots 76 retain stopcock 34 to flowpath element 30 without adhesive. Elimination of adhesive simplifies construction of DPT sub-assembly 26 by eliminating application of adhesive to mating surfaces of stopcock 34, flowpath connector 50, and flowpath element 30 and the associated adhesive cure time and equipment.

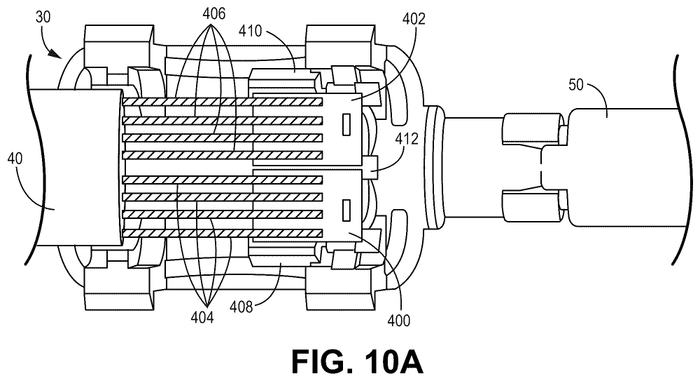

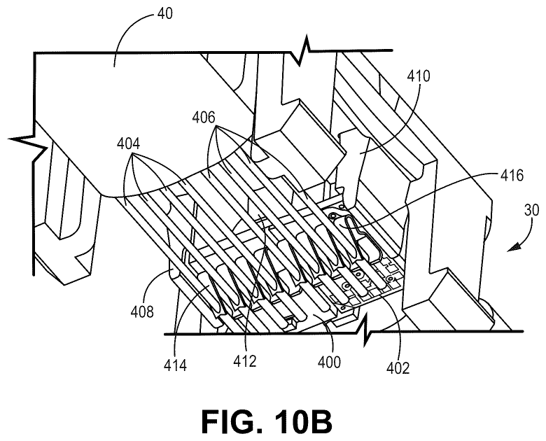

FIGS. 10A and 10B are broken-away plan and perspective views, respectively, of DPT sub-assembly 26 illustrating separate analog and digital sensors with dedicated sensor connectors. FIGS. 10A and 10B are described together and illustrate the attachment of connector cable 40 to sensors 108, supported by structural housing 58 (not shown; see FIGS. 5 and 6) against flowpath element 30. As noted previously, connector cable 40 contains multiple conductors 116. Although described generally as sensors 108, above, FIGS. 10A and 10B more specifically depict sensors 108 as including analog sensor 400 and digital sensor 402, contacted by analog and digital sensor conductors 404 and 406, respectively. As illustrated in FIGS. 10A and 10B, conductors 116 include four separate analog sensor conductors 404 and four separate digital sensor conductors 406, with each set including one ground conductor and three relative signal conductors. In alternative examples, however, more or fewer conductors may be used, as appropriate to the desired connector design and signal type.

As noted above, the multi-sensor architecture is described herein with reference to one analog and one digital differential pressure sensor, but can more broadly encompass any system with multiple separate, parallel differential pressure sensors disposed to generate signals of different types, carried separately within a common connector cable 40. As illustrated herein, analog sensor conductors 404 and digital sensor conductors 406 all lie in a common plane parallel to a separate common plane shared by analog sensor 400 and digital sensor 402. More generally, analog sensor conductors 404 lie in a plane parallel to and immediately adjacent analog sensor 400, and digital sensor conductors 406 analogously lie in a plane parallel to and immediately adjacent to digital sensor 402.

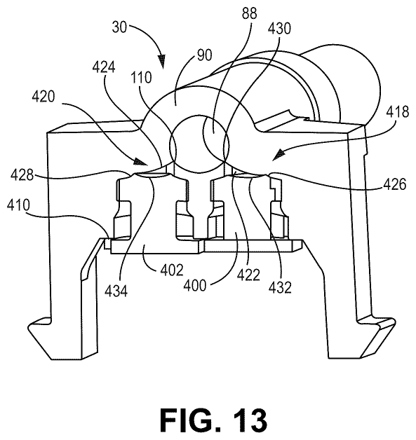

As depicted in FIGS. 10A and 10B, sensors 400 and 402 are positioned and retained by outer sensor spacing supports 408 and 410 and inner sensor spacing support 412 (shown in FIG. 10B). Outer sensor spacing supports 408 and 410 are flanged shelves with flanges extending laterally past outer edges of analog sensors 400 and 402, respectively, while inner sensor spacing support 412 is a central positioning support disposed at facing inner edges of analog sensors 400 and 402. Thus, analog sensor 400 is positioned and retained laterally between flanges of outer sensor spacing support 408 and inner sensor spacing support 412, while digital sensor 402 is similarly positioned and retained laterally between flanges of outer sensor spacing support 410 and inner sensor spacing support 412. Outer and inner sensor spacing supports 408, 410, and 412 cooperate with sensor support 114 of flowpath element 30 (see FIG. 6 and accompanying description) to lock both analog sensor 400 and digital sensor 402 in place relative to flowpath element 30, and thereby relative fluid channel 88 (see FIG. 6). More specifically, as described in greater detail below with reference to FIG. 13, sensor support 114 and inner and outer sensor spacing supports 408, 410, and 412 cooperate to position each sensor against a corresponding opening in flowpath element 30 to form separate fluidically sealed sensor cavities.

Contact pins 414 and 416 provide electrical contact between analog and digital sensors 400 and 402 and analog and digital sensor conductors 404 and 406, respectively. Contact pins 414 and 416 are omitted from FIG. 10A to better illustrate other components, but as illustrated in FIG. 10B are electrically isolated spring clips disposed in parallel and extending from their respective sensors to each respective conductor. More generally, contact pins 414 and 416 are separate electrical contacts between respective conductors and sensors, disposed with sensors 108 and conductors 116 in the sealed internal space between flowpath element 30 and structural housing 58 (not shown in FIG. 10A or 10B; see FIGS. 5 and 6).

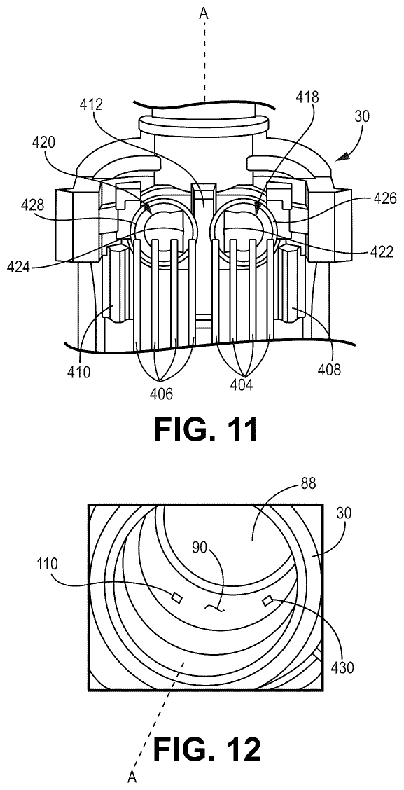

FIG. 11 is a bottom plan view of flowpath element 30 and conductors 404 and 406, with sensors 400 and 402 removed and sensor cavities visible. FIG. 11 illustrates outer centerline axis A and flowpath element 30 with sensor spacing supports 408 and 410 and inner sensor spacing support 412 as described above, as well as analog and digital sensor conductors 404 and 406, respectively. In addition, FIG. 11 depicts sensor cavity 418 positioned to abut analog sensor 400, and sensor cavity 420 positioned to abut digital sensor 402.

Cavities 418 and 420 include flowpath connections 422 and 424, respectively, and sensor abutments 426 and 428, respectively. As depicted in FIG. 11, sensor cavities 418 and 420 are recesses in flowpath element 30 disposed between the retention positions of sensors 402 and 404, respectively, and fluid channel 88. As illustrated in FIG. 11, sensor abutments 426 and 428 are circular perimeters of sensor cavities 418 and 420 immediately abutting analog and digital sensors 400 and 402, respectively. More generally, sensor abutments 426 and 428 are mating openings facing analog and digital sensors 400 and 402, respectively. As described in greater detail below with respect to FIG. 13, sensors 400 and 402 are installed flush against sensor abutments 426 and 428, respectively, such that sensor cavities 418 and 420 form fluid seals with their respective sensors 400 and 402 surrounding fluid-facing membranes of each sensor, and each cavity cooperates with its respective sensor to enclose a respective fluid chamber adjacent to and only fluidly connected to fluid channel 88. Flowpath connections 422 and 424 are passages preferably narrow, but can in the most general example each have angular widths up to approximately 25% of the circumference of fluid channel 88 at their axial location. In an illustrative embodiment, flowpath connections 422 and 424 can have circumferential widths of at least 0.005 inches. Flowpath connections 422 and 424 have equal axial lengths of at least 0.005 selected to permit reliable molding, i.e. to avoid closure of flowpath connections 422 or 424 during part molding. Flowpath connections 422 and 424 have axial length no greater than an axial extent of a sealing surface of sensors 400 and 402. In an illustrative embodiment, flowpath connections 422 and 424 can have an axial length of 0.07 to 0.08 inches. Flowpath connections 422 and 424 are sized to allow fluid communication between channel 88 and both sensors 400 and 402, while reducing or minimizing bubble formation in fluid channel 88.

FIG. 12 illustrates the intersection of flowpath connections 422 and 424 (not separately shown in FIG. 12) with fluid channel 88. FIG. 12 is a perspective view of the interior of flowpath element 30, askew with respect to centerline axis A, with fluid channel 88 defined by channel wall 90. As depicted in FIG. 12, sensor cutouts 110 (see FIG. 6) and 430 are apertures in channel wall 90 that define the intersections of flowpath connections 422 and 424, respectively with channel 88.

FIG. 13 is a cross-sectional perspective view of flowpath element 30 through a plane transverse to centerline axis A, illustrating the positioning of both sensors 400 and 402 and respective cutouts relative to fluid channel 88. FIG. 13 also depicts sensor cavities 418 and 420, which include flowpath connections 422 and 424, sensor abutments 426 and 428, and sensor cutouts 430 and 110, respectively. Sensor faces 432 and 434 of sensors 400 and 402, respectively, are also shown.

As mentioned above with reference to FIGS. 11 and 12, sensors 400 and 402 have sensor faces 432 and 434 in fluid contact with IV fluid flowing through fluid channel 88, within flowpath element 30. Sensor faces 432 and 434 can, for example, be deformable pressure-sensitive membranes. Sensor cavities 418 and 420 in flowpath element 30 cooperate with sensor faces 432 and 434, respectively, to define sensor chambers fluidly connected to fluid channel 88 through sensor cutouts 430 and 110, respectively, at identical axial locations relative to centerline axis A (see, e.g., FIGS. 11 and 12). In the illustrated example, sensor cutouts 110 and 430 are separated by an angle of approximately 135° relative to centerline axis A. More generally, sensor cutouts 110 and 430 can be separated by an angle greater than 90° and less than 180°, such that fluid is able to flow directly and linearly from channel 88 to sensor faces 432 and 434, and such that bubble formation is minimized.

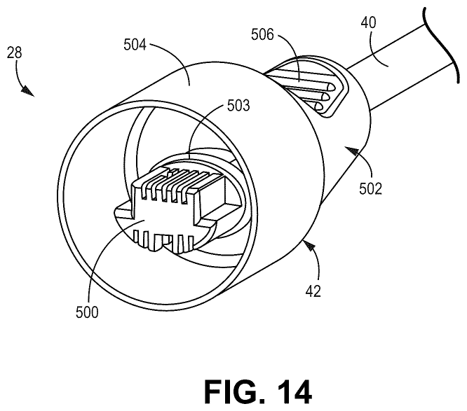

FIG. 14 is perspective view of hybrid connector 28 illustrating a distal end of connector cable 40 terminating at signal connector 42. Signal connector 42 includes plug 500, overmolding 502, seal ring 503, and shroud 504. Overmolding 502 includes grip 506.

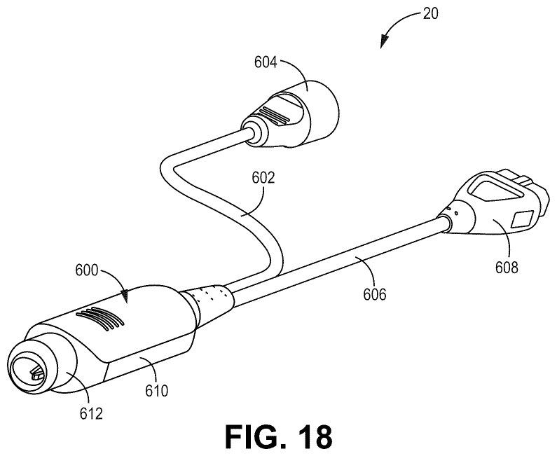

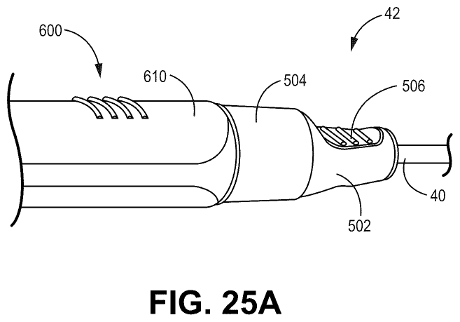

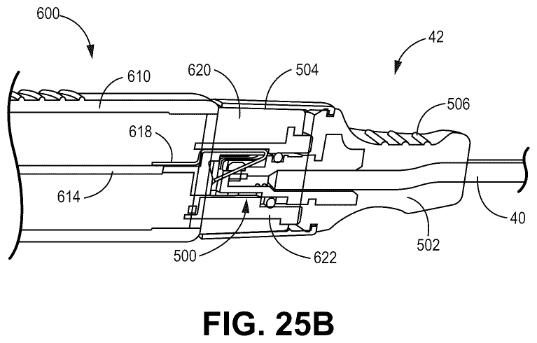

Signal connector 42 is an electrical connector sized and shaped to mate to a corresponding receptacle (see FIGS. 18-25B, described below) to transmit alternative or multiple types of electrical signals through a single mechanical connection. Plug 500 is a rigid insert received in the receptacle, while shroud 504 is a deformable cover generally cylindrically surrounding but spaced apart from plug 500, so as to surround and abut the receptacle while plug 500 is installed. The connection of plug and receptacle are described in greater detail below with reference to FIGS. 25A and 25B. Shroud 504 both helps to secure plug 500 in the receptacle and forms a partial fluid seal preventing fluid ingress to electrical contacts of signal connector 42 while installed at a receptacle. In the illustrated example, shroud 504 is a separate component attached to overmolding 502, a flexible or deformable base that surrounds a distal end of connector cable 40. In some alternative examples, however, overmolding 502 and shroud 504 may be functional components of a single protective component. Overmolding 502 and shroud 504 can, for example, be flexible polymer component such as a rubber or rubberized sheath. As illustrated in FIG. 14, overmolding 502 includes grip 506, a ridged, deformable section provided at a proximal end of signal connector 42 to facilitate gripping signal connector 42 to install or remove plug 500 from an appropriate receptacle. Seal ring 503 is a ring of compressible material such as an O-ring, and both helps to retain plug 500 in position in a complementary receptacle and protects electrical components from exposure to moisture when plug 500 is installed.

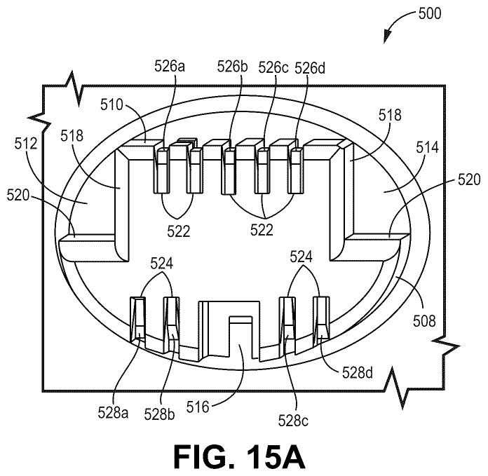

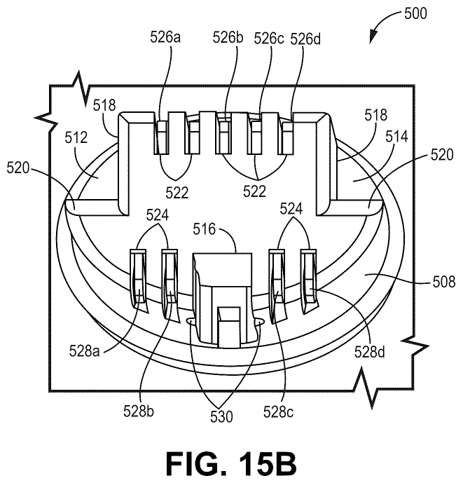

FIGS. 15A and 15B illustrate plug 500 in greater detail. FIGS. 15A and 15B are close-up perspective views illustrating oval perimeter 508, top wall 510, cutouts 512 and 514, keyway 516, vertical and horizontal walls 518 and 520, respectively, top pin slots 522, bottom pin slots 524, analog signal pins 526a-d (collectively referred to as analog signal pins 526), and digital signal pins 528a-d (collectively referred to as digital signal pins 528). FIGS. 15A and 15B differ only in view angle, with FIG. 15A providing an oblique downward view of plug 500 for clearer depiction of top wall 510 and analog signal pins 526, and with FIG. 15B providing an oblique upward view of plug 500 for clearer depiction of keyway 516 and digital signal pins 528.

As noted above, plug 500 is shaped to fit into a mating receptacle, thereby pressing analog signal pins 526, digital signal pins 528, or both against corresponding electrical contacts. Analog and digital signal pins provide electrical contacts for pressure signals from analog and digital sensors 400 and 402, respectively. By providing both digital and analog sensor connections in a single plug, signal connector 42 allows DPT sub-assembly 26 to be freely connected to digital and analog monitors, without need for additional hardware. In the illustrated example, analog signal pins 526 are arranged in a row along top wall 510, while digital signal pins 528 are arranged in a parallel row, disposed opposite analog signal pins 526. Analog signal pins 526 face, i.e. are exposed, on distal and upward (with reference to the views of FIGS. 15A and 15B) faces, while digital signal pins 528 face, i.e. are exposed, on distal and downward faces.

Cutouts 512 and 514 and keyway 516 are alignment and retention features that facilitate easy alignment and insertion of plug 500 to an appropriate receptacle, while preventing misalignments such as reversals of positions of digital and analog signal pins 526, 528. The specific form factor of plug 500 thus complements its intended receptacle to receive and securely retain plug 500. Specifically, the overall size and shape of plug 500 are principally described by oval perimeter 508, depicted in FIGS. 15A and 15B as an oval or rounded oblong cross-sectional shape with a major (i.e. longest) axis generally parallel to the rows of both analog signal pins 526 and digital signal pins 528. The outer perimeter of plug 500 as defined by oval perimeter 508 is interrupted by top wall 510, cutouts 512 and 514, and keyway 516. In an exemplary embodiment, oval perimeter 508 can have a major axis no greater than 0.5 inches, and a minor axis no greater than 0.37 inches to permit engagement with a corresponding oval socket (discussed subsequently).

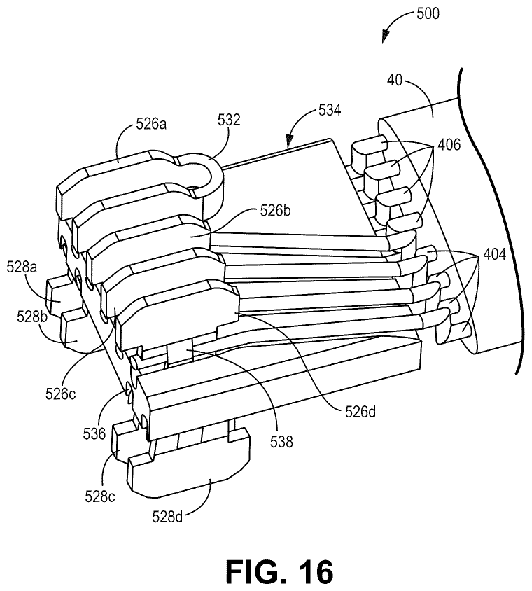

Top wall 510 is a flat wall at a top (relative to FIGS. 15A and 15B) portion of plug 500. Top pin slots 522 are spaced—evenly, in the illustrated example—across top wall to provide access to analog signal pins 526. In the most general case, at least as many top and bottom pin slots 522, 524 are present as analog and digital signal pins 526, 528, respectively. The example provided in FIGS. 15A and 15B includes four digital signal pins 528, each disposed in a respective bottom pin slot 524, and four analog signal pins 526, with five top pin slots 522. In this example, one analog signal pin 526 (specifically, analog signal pin 526a) is a U-shaped pin that occupies two adjacent top pin slots. This U-shaped geometry can be seen in greater detail in FIG. 16. By contrast with analog signal pins 526, which are evenly offset from top wall 510 via top pin slots 522, digital signal pins 528 are variously offset from oval perimeter 508 by bottom pin slots 524 of multiple depths so as to fall in a line. As illustrated in FIGS. 15A and 15B, digital signal pins 528a and 258d are recessed in shallow pin slots, while digital signal pins 528b and 528c are recessed in relatively deeper pin slots.

FIGS. 15A and 15B illustrates cutouts 512 and 514 as angled (specifically, right-angled) recesses into oval perimeter 508. More generally, cutouts 512 are features within the form factor defined by oval perimeter 508 that mate to corresponding receptacle shelves (see FIGS. 18-24, described below) to retain and align plug 500. Similarly, keyway 516 is a slot through a bottom portion of plug 500, between digital signal pins 528b and 528c, that both aligns plug 500 and gently locks plug 500 into place when installed. Specifically, keyway 516 includes snap slot 530 (shown in FIG. 15B), a laterally widened axial section within keyway 516 disposed to receive a spring-deformable mating receptacle component to prevent plug 500 from being unintentionally dislodged from a mated receptacle.

The particular shape of plug 500 distributes multiple analog and digital signal pins 526, 528 across a small form factor while ensuring proper alignment of both sets of pins with corresponding electrical contracts and retaining plug 500 in its installed position. As described below, the exterior form factor of plug 500 matches an internal geometry of the receptacle (612; see e.g. FIG. 18).

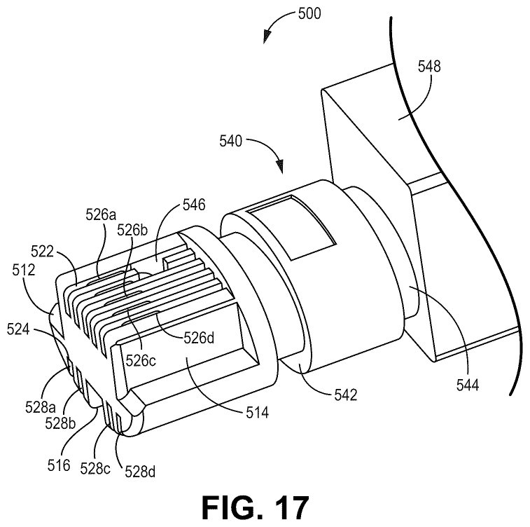

FIGS. 16 and 17 are perspective views of connector plug 500 illustrating internal components thereof. FIG. 16 illustrates internal wires, pins, and a wire guide of plug 500, while FIG. 17 illustrates the positioning of the form factor-defining modular plug around these internal components.

FIG. 16 depicts connector cable 40 containing analog signal conductors 404 and digital signal conductors 406 as described above, as well as analog signal pins 526 and digital signal pins 528. In addition, FIG. 16 illustrates pin U-turn 532, wire guide 534 (with conductor slots 536), and pin teeth 538.

As previously noted, in some examples one or more pins can be co-located in multiple pin slots. Pin U-turn 532 is an example turn in analog signal pin 526a such that it is disposed in multiple slots, for contacting one or multiple corresponding contacts of a mated receptacle. Although only one pin U-turn is illustrated, and although pin U-turn 532 is the only illustrated example of one pin disposed in multiple slots, other examples can include any number of multi-slot pins appropriate to expected number of electrical contacts.

Wire guide 534 is a supporting structure that separates and retains analog signal conductors 404 and digital signal conductors 406. More specifically, as illustrated in FIG. 16, wire guide 534 includes multiple conductor slots 536—one slot for each conductor. Wire guide 534 is disposed between analog signal conductors 404 and digital signal conductors 406, with analog signal conductors 404 being retained in conductor slots 536 in a top surface of wire guide 534 (with reference to the orientation of FIG. 16) and digital signal conductors 406 being retained in conductor slots 536 in a bottom surface of wire guide 534. Conductor slots 536 not only receive, and retain signal conductors 404, 406, but also serve to position signal conductors 404, 406 relative to digital and analog signal pins 526, 528 and top and bottom pin slots 522, 524. Each analog or digital signal pin 526, 528 has multiple pin teeth 538 that capture and electrically contact one signal conductor 404, 406. Pin teeth 538 are embedded and retained within conductor slots 536, thereby securing distal ends of all analog and digital signal conductors 404, 406 within plug 500.

FIG. 17 provides a perspective view of modular plug 540 surrounding wire guide 534 (not shown in FIG. 17), with other illustrated elements generally as described above. Modular plug 540 is a protective cover that defines the previously described external shape of plug 500, including cutouts 512 and 514, keyway 516, top pin slots 522, and bottom pin slots 524. In addition, modular plug 540 includes seal ring slot 542 to accommodate and receive seal ring 503 (shown in FIG. 14), and overmolding slot 544 to anchor overmolding 502 (shown in FIG. 14) relative to plug 500. Modular plug 540 also includes accommodating gap 546 disposed between adjacent top pin slots 522 for analog signal pin 526a to permit pin U-turn 538. The proximal end of modular plug 540, relative to DPT sub-assembly 26, includes connector cap 548, a fitting that contains the terminal end of conductor cable 40 within modular plug 540. Overall, modular plug 540 captures analog and digital conductors 404, 406 and prevents undesirable electrical contacts with all conductors, accommodates and positions analog and digital signal pins 526 and 528, defines retaining and alignment features for plug 500 as a whole (i.e. cutouts 512, 514 and keyway 516), and seals the terminal end of connector cable 40. During assembly, pins 526, 528 are installed after wire guide 534 and signal conductors 404, 406 have been inserted into modular plug 540, through top and bottom pin slots 522, 524, thereby serving a further retaining function for modular plug 540.

FIG. 18 is a perspective view of multi-signal pressure cable 20, as previously introduced in FIG. 2. Multi-signal pressure cable 20 includes hybrid connector 600 adapted to receive plug 500. Analog signal cable 602 connects hybrid connector 600 to analog signal connector 604, and digital signal cable 606 connects hybrid connector 600 to digital signal connector 608. Hybrid connector 600 includes outer shell 610 and receptacle 612.

As noted above, hybrid connector 600 includes separate contacts disposed to independently receive both analog signals from analog sensor 400 and digital signals from digital sensor 402. As shown in FIG. 18, hybrid connector 600 serves as a common signal input with split outputs. Specifically, analog signal connector 604 carries analog signals received at hybrid connector 600 to a complementary device expecting analog pressure signals, while digital signal connector 608 analogously carries digital signals received at hybrid connector 600 to a complementary device expecting digital pressure signals. As analog and digital signal generation and transmission are independent according to the examples provided herein, multi-signal pressure cable 20 can be connected to a digital monitor (e.g. primary monitor 22; see FIG. 2), an analog monitor (e.g. secondary monitor 24; see FIG. 2), or both simultaneously. The connector types of analog and digital signal connectors 604 and 606 can have any shapes appropriate to mate to expected monitoring equipment. Receptacle 612 is a plug receiver disposed at a combined distal end of both analog signal connector 604 and digital signal connector 608. Outer shell 610 is a protective housing, e.g. a clamshell assembly, situated immediately proximal of receptacle 612, between receptacle 612 and signal connectors 604, 608, to protect internals of hybrid connector 600.

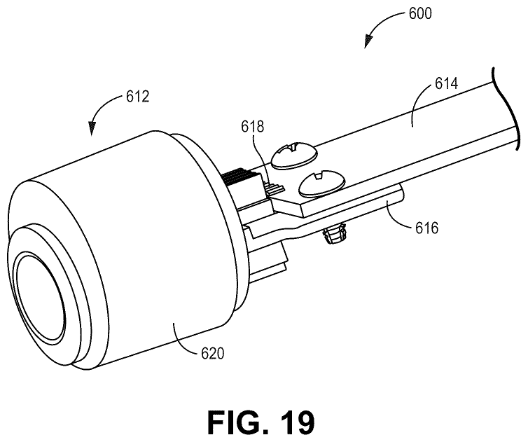

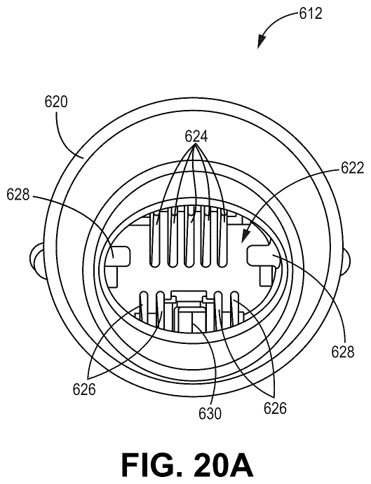

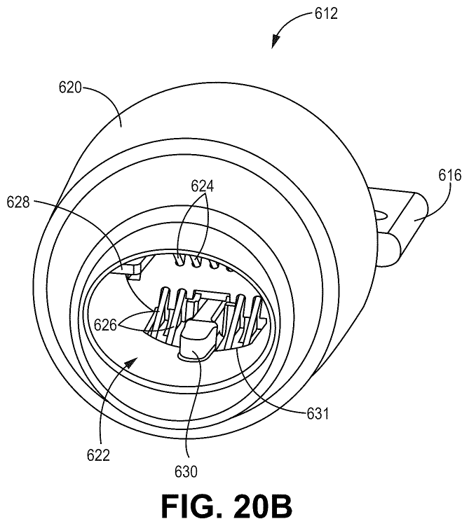

FIG. 19 is a perspective view of components of hybrid connector 600, including receptacle 612, circuit board 614, structural anchor 616, electrical connections 618, and receptacle overmold 620. Circuit board 614, structural anchor 616, and electrical connections 618 are internal components of hybrid connector 600 surrounded and protected by outer shell 610 (see FIG. 18). FIGS. 20A and 20B are perspective and front views, respectively, into receptacle 612 of hybrid connector 600. FIGS. 19, 20A, and 20B are described together.

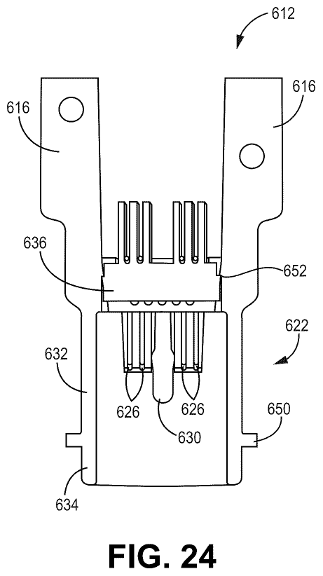

Receptacle 612 is configured to receive plug 500 as noted above and described in greater detail below with reference to FIGS. 25A and 25B. Electrical connections 618 routing to analog signal connector 604 and digital signal connector 606 are handled within and/or attached to circuit board 614. Circuit board 614 is rigidly connected to receptacle 612 via at least one structural anchor 616 as described in greater detail below with reference to FIG. 24. Structural anchor 616 can be a separate connector, or an extension of other elements of receptacle 612 as described subsequently.

Receptacle 612 includes receptacle overmold 620 surrounding oval socket 622. Oval socket 622 is a rigid structural component defining the interior shape of receptacle 612, as sized and shaped to receive plug 500. Specifically, oval socket 622 includes retention and alignment features that align with complementary features of plug 500, including shelves 628 that align with cutouts 512 and 514, and snap key 630 that is received via a snap fit within keyway 516. The overall interior shape of oval socket 622 has a substantially oval cross-section generally matching oval perimeter 508 of plug 500, to snugly receive plug 500 therewithin. In the example illustrated in FIG. 20A, shelves 628 are separate platforms on a common plate generally parallel and close to a major axis of the oval cross-section of oval socket 622. More generally, shelves 628 are geometric keys matching the corresponding shapes of alignment and retention features of plug 500, such as cutouts 512 and 514. As depicted in FIG. 20B, snap key 630 is an axially extending protrusion along the bottom (with respect to the viewing angles of FIGS. 20A and 20B) interior of oval socket 622 and having a widened section that compresses slightly to snap fit to snap slot 530 of keyway 516 (see FIG. 15B, described above). As illustrated in FIGS. 20A and 20B, the exterior cross-section of oval socket 622 is circular, in contrast to its oval internal cross-section.

Analog signal contacts 624 and digital signal contacts 626 are electrical contacts extending through an interior oval wall of oval socket 622 via contact openings 631 to engage analog and digital signal pins 526 and 528, respectively. The number and positioning of analog signal contacts 624 and digital signal contacts 626 can match the number and positioning of analog and digital signal pins 526 and 528, respectively. Alternatively, as shown in FIG. 20B, the number of contacts may be varied, e.g. to match multiple contacts to complex pins such as analog signal pin 528a with its U-shaped geometry. Analog and digital signal contacts 624 and 626 are depicted and described further below.

Receptacle overmold 620 is a pliable or deformable cover surrounding and protecting oval socket 622. Receptacle overmold 620 can, in some examples, provide a seal against fluid ingress to electrical components of hybrid connector 600. Receptacle overmold can have a rounded or chamfered cylindrical shape, or a tapered or frustoconical shape as illustrated in FIGS. 20A and 20B, surrounding but offset from the center of the oval internal cavity defined by oval socket 622.

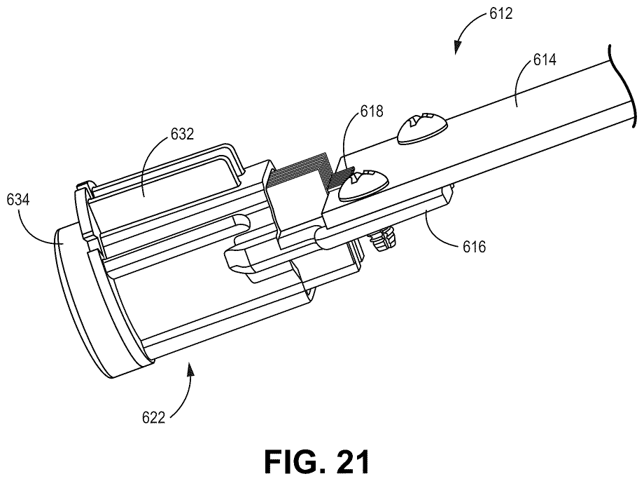

FIGS. 21, 22A, 22B, 23, and 24 are perspective views of stripped-down or broken-away sections of hybrid connector 600 illustrating internal components. FIG. 21 depicts parts generally as described above with respect to FIGS. 19, 20A, and 20B, but omits overmold 620 to expose further portions of oval socket 622 including structural container 632 and receptacle face 634. As illustrated in FIG. 21, structural anchor or anchors 616 can be integral plates or extension of oval socket 622 extending parallel to and abutting circuit board 614. In contrast to its smooth and tapered internal geometry adapted to receive plug 500, the exterior of oval socket 622 can, as illustrated in FIG. 21, include ridges, flanges, and notches disposed to anchor and prevent rotation of receptacle overmold 620 relative to oval socket 622.

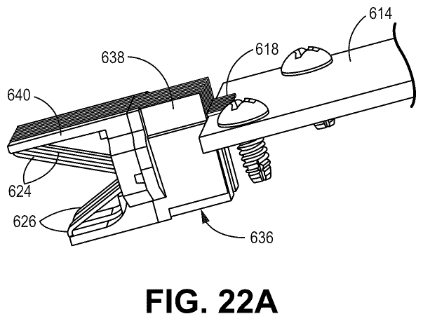

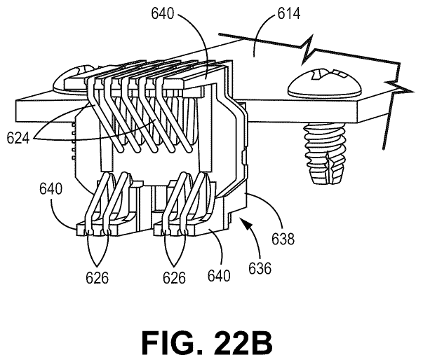

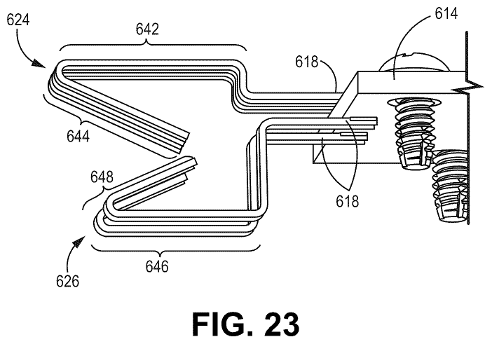

FIGS. 22A and 22B illustrate components of receptacle 612 substantially as described above with reference to FIG. 21, but omit overmold 620 to expose contact support 636 with contact support body 638 and contact support arms 640. FIG. 23 further omits contact support 636 to illustrate the shape and internal positioning of analog signal contacts 624 and digital signal contacts 626, including as they contact circuit board 614 via electrical connections 618. Contact support 636 is a rigid structure housed within oval socket 622 and outer shell 610, and configured to separate, position, and support analog signal contacts 624 and digital signal contacts 626. Contact support 636 includes a contact support body situated between circuit board 614 and a back wall of the interior chamber of oval socket 622. Contact support 636 also includes multiple contact support arms 640 acting as shelves extending distally away from contact support body 638 and engaging analog and digital signal contacts 624, 626. In the illustrated example, contact support arms 640 are housed within the upper and lower (with reference to the orientations of FIGS. 20A and 20B) spaces defined between the oval cross-sectioned interior wall of oval socket 622 and its generally cylindrical outer wall. More specifically, as best seen in FIG. 22B, contact support 636 includes one contact support arm 640 abutting the row of analog signal contacts in position to engage analog signal pins 526, and two separate contact support arms 640 supporting sets of digital signal contacts 626 disposed on either side of snap key 634.

As seen in FIG. 23, analog signal contacts 624 and digital signal contacts 626 both attach to circuit board 614 via electrical connections 618. Each analog signal contact 624 includes electrical contact section 644 and axial section 642 that turns inward and backward towards digital signal contacts 626 and circuit board 614. Similarly, each digital signal contact 626 includes electrical contact section 648 and axial section 646 that turns inward and backward towards analog signal contacts 624 and circuit board 614. This arrangement spring-loads electrical contact sections 644 and 648 of analog and digital signal contacts 624 and 626, respectively, into engagement with pins of plug 500.

FIG. 24 provides a further cross-sectional view of elements of receptacle 612 and through a plane disposed between analog signal contacts 624 and digital signal contacts 626. FIG. 24 illustrates oval socket 622 with structural container 632 as previously described, cylindrically surrounding digital signal contacts 626 and snap key 630. Oval socket 622 also includes receptacle face 634 and overmold retaining flange 650. Overmold retaining flange 650 provides an anchor for receptacle overmold 620 (not shown; see FIGS. 19-21). In some examples, receptacle face 634 can extend distally past an axial extent of receptacle overmold 620, while in other examples receptacle face 634 can be captured at least partially within receptacle overmold 620. As depicted in FIG. 24, oval socket 622 includes two separate integral structural anchors 616 fastened to circuit board 614 (see FIG. 21). More generally, any number or shape of structural anchors 616 can be used that rigidly attaches oval socket 622, and thereby contact support 634 and analog and digital signal contacts 624 and 626, to circuit board 614. FIG. 24 also illustrates snap engagement 652 between contact support 636 and oval socket 622, which retains contact support body 638 against a rear wall of oval socket 622.

FIGS. 25A and 25B are simplified perspective and cross-sectional perspective views, respectively, of a plug 500 engaging hybrid connector 600. When engaged, as described above, plug 500 is inserted within oval socket 622, thereby forcing analog signal pins 526 into electrical contact with analog signal contacts 624, and digital signal pins 528 into electrical contact with digital signal contacts 626. Snap key 630 engages keyway 516 in a snap fit, oval perimeter 508 of plug 500 smoothly abuts the interior surface of oval socket 622, and shroud 504 overlaps receptacle overmold 620 to create a durable, fluid-sealed connection. A row of electrical contact sections 644 of analog signal contacts 624 engage analog signal pins 624 from above, while a row of electrical contact sections 648 of digital signal contacts 626 engage digital signal pins 626 from below, permitting differential pressure signals from both analog sensor 400 and digital sensor 402 to be received and transmitted to analog signal connector 604 and digital signal connector 608, respectively, and from there to appropriate patient monitors expecting digital and/or analog pressure signals.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### DISCUSSION OF POSSIBLE EXAMPLES

The following are non-exclusive descriptions of possible examples of the present invention.

A dual differential pressure transducer assembly comprising: an intravenous (IV) fluid inlet configured to receive fluid from an IV bag; an IV fluid outlet configured to supply the fluid to a patient IV needle; a flowpath element defining a fluid channel connecting the IV fluid inlet to the IV fluid outlet, the fluid channel defining a centerline axis of the dual differential pressure transducer assembly; a first sensor cavity disposed alongside the fluid channel and fluidly connected to the fluid channel via a first cutout through the flowpath element, the first cutout extending transverse to the centerline axis; a second sensor cavity disposed alongside the fluid channel, parallel with the first sensor cavity, and fluidly connected to the fluid channel via a second cutout through the flowpath element, the second cutout extending transverse to the centerline axis, and axially aligned but angularly offset from the first cutout, relative to the centerline axis; a first differential pressure sensor having a first sensor face abutting the first sensor cavity and exposed to the fluid channel via the first cutout; a second differential pressure sensor having a second sensor face abutting the second sensor cavity and exposed to the fluid channel via the second cutout; and separate signal conductors connected to the first and second differential pressure sensors, respectively.

The dual differential pressure transducer assembly of the preceding paragraph can optionally include, additionally and/or alternatively, any one or more of the following features, configurations and/or additional components:

A further embodiment of the foregoing dual differential pressure transducer assembly, wherein the separate signal conductors comprise: a first plurality of signal conductors disposed to carry analog electrical signals from the first differential pressure sensor; and a second plurality of signal conductors disposed to carry digital signals from the second differential pressure sensor, wherein the first and second pluralities of signal conductors are arranged in a common plane alongside the first and second sensors, and enclosed together with the first and second differential pressure sensors within a common housing.

A further embodiment of the foregoing dual differential pressure transducer assembly, wherein the common housing surrounds the first differential pressure sensor, the second differential pressure sensor, and at least a portion of the fluid channel aligned with the first and second cutouts.

A further embodiment of the foregoing dual differential pressure transducer assembly, wherein the first and second cutouts are separated by an angle less than 180°, relative to the centerline axis.

A further embodiment of the foregoing dual differential pressure transducer assembly, wherein the first and second cutouts are separated by an angle greater than 90°, relative to the centerline axis.

A further embodiment of the foregoing dual differential pressure transducer assembly, wherein the fluid channel has a diameter between 1 and 8 mm at the axial location of the first and second cutouts.

A further embodiment of the foregoing dual differential pressure transducer assembly, wherein a cross-sectional area of the fluid channel through planes orthogonal to the centerline axis is narrowest at a location upstream of the first and second cutouts.

A further embodiment of the foregoing dual differential pressure transducer assembly, wherein the first and second cutouts intersect the fluid channel at first and second channel apertures.

A further embodiment of the foregoing dual differential pressure transducer assembly, wherein the first and second channel apertures each have an angular extent less than 5° with respect to the centerline axis.

A further embodiment of the foregoing dual differential pressure transducer assembly, wherein the first and second cutouts each widen as a function of radial position with respect to the centerline axis, from narrowest at the first and second channel apertures, respectively, to widest at the first and second sensor faces, respectively.

A further embodiment of the foregoing dual differential pressure transducer assembly, wherein the first and second cutouts are each defined in part by walls extending from the fluid channel to the first and second differential pressure sensors, respectively, and wherein the walls are parallel to but offset from each other and the centerline axis.

A further embodiment of the foregoing dual differential pressure transducer assembly, wherein the first and second channel apertures are elongate apertures with major dimensions parallel to the centerline axis.

A further embodiment of the foregoing dual differential pressure transducer assembly, wherein the first and second channel apertures each have a circumferential width relative to the centerline axis that is greater than 0.005 inches (0.133 mm).

A further embodiment of the foregoing dual differential pressure transducer assembly, wherein the first and second channel apertures each have a circumferential width relative to the centerline axis that is less than 25% of the total circumference of the channel at locations of the first and second channel apertures.

A further embodiment of the foregoing dual differential pressure transducer assembly, wherein the first and second channel apertures each have an axial length, relative to the centerline axis, of at least 0.005 inches, and less than an axial extent of sealing surfaces of the first and second differential pressure sensors.

A further embodiment of the foregoing dual differential pressure transducer assembly, wherein the first and second channel apertures each have an axial length, relative to the centerline axis, of between 0.07 and 0.08 inches.

A further embodiment of the foregoing dual differential pressure transducer assembly, wherein at least one of the first and second sensor faces is defined by a deflecting gel diaphragm.

A further embodiment of the foregoing dual differential pressure transducer assembly, further comprising a stopcock disposed along the fluid channel, downstream of the first and second cutouts, and actuatable between multiple valve states, including: a first state fluidly separating the IV fluid inlet from the IV fluid outlet; and a second state fluidly connecting the IV fluid inlet to the IV fluid outlet.

A further embodiment of the foregoing dual differential pressure transducer assembly, wherein the stopcock is a three-state valve.

A further embodiment of the foregoing dual differential pressure transducer assembly, further comprising a flush tab disposed alongside a wall within the fluid channel and selectively permit flow past the wall, from upstream to downstream through the fluid channel.

A further embodiment of the foregoing dual differential pressure transducer assembly, wherein the flush tab is disposed upstream of the first and second cutouts.

A further embodiment of the foregoing dual differential pressure transducer assembly, wherein the dual differential pressure transducer assembly is sterilized.

A further embodiment of the foregoing dual differential pressure transducer assembly, further comprising a connector cable terminating at a connector plug and surrounding the separate signal conductors from the first and second differential pressure sensors to the connector plug.

A further embodiment of the foregoing dual differential pressure transducer assembly, wherein the connector plug comprises: a plug body disposed to be inserted in a receiving receptacle; a first plurality of pins electrically connected to first differential pressure sensor via a first subset of the separate signal conductors; and a second plurality of pins electrically connected to the second differential pressure sensor via the second subset of the separate signal conductors separate from the first subset of the separate signal conductors; wherein the first and second pluralities of pins are disposed on opposite sides of the plug body.

A differential pressure transducer assembly comprising: an intravenous (IV) fluid channel; a first differential pressure sensor disposed to generate a first sensor signal indicative of fluid flow through the IV fluid channel; a first plurality of signal conductors electrically connected to the first differential pressure sensor to carry the first sensor signal; a second differential pressure sensor disposed to generate a second sensor signal indicative of fluid flow through the IV fluid channel, the second sensor signal being different from the first sensor signal; a second plurality of signal conductors electrically connected to the second differential pressure sensor to carry the second sensor signal; a connector cable extending from the first and second differential pressure sensors and containing the first and second pluralities of signal conductors; and a connector plug terminating the connector cable and defining a plug axis, the connector plug comprising: a first plurality of pins electrically connected to the first plurality of signal conductors; a second plurality of pins electrically connected to the second plurality of signal conductors; and a wire guide separating the first plurality of signal conductors from the second plurality of signal conductors at the connector plug, and retaining and guiding the first and second pluralities of signal conductors to the first and second pluralities of pins, respectively.

The differential pressure transducer assembly of the preceding paragraph can optionally include, additionally and/or alternatively, any one or more of the following features, configurations and/or additional components:

A further embodiment of the foregoing differential pressure transducer assembly, wherein the first and second differential pressure sensors are analog and digital sensors, respectively, such that the first sensor signal is an analog signal and the second sensor signal is a digital signal.

A further embodiment of the foregoing differential pressure transducer assembly, further comprising a substantially cylindrical or frustoconical shroud disposed about the plug axis, coaxially about the connector plug.

A further embodiment of the foregoing differential pressure transducer assembly, further comprising a grip affixed to the connector cable and the shroud.

A further embodiment of the foregoing differential pressure transducer assembly, wherein the grip is a resilient overmolding.

A further embodiment of the foregoing differential pressure transducer assembly, wherein the connector plug further comprises a modular plug surrounding and capturing the wire guide while exposing both the first and second pluralities of pins.

A further embodiment of the foregoing differential pressure transducer assembly, wherein the first and second pluralities of pins are disposed on opposite sides of the modular plug.

A further embodiment of the foregoing differential pressure transducer assembly, wherein all of the first and second pluralities of pins are insertable through the modular plug, into the wire guide, to form electrical connections with the first and second pluralities of pins, respectively.

A further embodiment of the foregoing differential pressure transducer assembly, wherein the modular plug has an oval or elliptical cross-section through at least one cross-section in a plane orthogonal to the plug axis, the oval or elliptical cross-section defining defined by a major axis, such that opposite first and second portions of the modular plug are separated by the major axis of the oval or elliptical cross-section.

A further embodiment of the foregoing differential pressure transducer assembly, further comprising a fluid sealing element disposed about the modular plug.

A further embodiment of the foregoing differential pressure transducer assembly, wherein modular plug includes a plurality of alignment features configured to prevent misalignment of the connector plug with an improper receptacle, or at a nonfunctional angle.

A further embodiment of the foregoing differential pressure transducer assembly, wherein the alignment features comprise angular cutouts from the perimeter of the modular plug, the angular cutouts defined by surfaces substantially parallel to the plug axis.

A further embodiment of the foregoing differential pressure transducer assembly, wherein there are two angular cutouts, and wherein the angular cutouts are situated entirely within the first portion and partially defined by parallel surfaces that symmetrically bracket a center portion of the modular plug.

A further embodiment of the foregoing differential pressure transducer assembly, wherein the alignment features further comprise a keyway disposed symmetrically with respect to the two angular cutouts, within the second portion.

A further embodiment of the foregoing differential pressure transducer assembly, wherein the keyway extends axially for a subset of an axial length of the modular plug, and flares to a maximum width proximate an axially central location of the subset of the axial length.

A further embodiment of the foregoing differential pressure transducer assembly, wherein the first plurality of pins are disposed in and exposed through the first portion of the modular plug, and the second plurality of pins are disposed in and exposed through the second portion of the modular plug.

A further embodiment of the foregoing differential pressure transducer assembly, wherein the first and second pluralities of pins are exposed axially and laterally through the modular plug.

A further embodiment of the foregoing differential pressure transducer assembly, wherein each of the first and second pluralities of pins electrically contacts exactly one of the first and second pluralities of signal conductors, respectively.

A further embodiment of the foregoing differential pressure transducer assembly, wherein at least one of the first plurality of pins is a U-shaped double pin.

A further embodiment of the foregoing differential pressure transducer assembly, wherein the first and second pluralities of signal conductors are equal in number.

A further embodiment of the foregoing differential pressure transducer assembly, wherein at least one of each of the first and second pluralities of signal conductors corresponds to a ground.

A sensor signal connector configured to separately carry sensor signals from a first sensor and a second sensor, the sensor signal connector comprising: a first plurality of signal conductors electrically connected to the first sensor; a second plurality of signal conductors electrically connected to the second sensor; a connector cable surrounding the first and second pluralities of signal conductors; a wire guide disposed at an end of the connector cable and having opposite first and second sides, the wire guide disposed to retain exposed ends of the first plurality of signal conductors at connection locations on the first side, and to retain exposed ends of the second plurality of signal conductors on the second end; a plurality of pins each attached to one of the first or second pluralities of signal conductors and secured in the wire guide; and a modular plug defining an outer form factor of the sensor signal connector securable in a sensor receptacle, the modular plug surrounding the wire guide, securing the first and second pluralities of signal conductors to the wire guide, and exposing the plurality of pins.

The sensor signal connector of the preceding paragraph can optionally include, additionally and/or alternatively, any one or more of the following features, configurations and/or additional components:

A further embodiment of the foregoing sensor signal connector, wherein each of the plurality of pins has a chamfered or beveled edge furthest from the wire guide.

A further embodiment of the foregoing sensor signal connector, wherein at least one of the first and second pluralities of signal conductors corresponds to a ground.

A further embodiment of the foregoing sensor signal connector, wherein the wire guide and the connector cable are formed of insulating material, and the plurality of pins are formed of conductive material.

A further embodiment of the foregoing sensor signal connector, wherein all of the first and second pluralities of signal conductors are arranged in a plane within the connector cable.

A further embodiment of the foregoing sensor signal connector, wherein the wire guide separates and aligns the first plurality of signal conductors in a first plane on the first side, and the second plurality of signal conductors in a second plane on the second side.

A further embodiment of the foregoing sensor signal connector, wherein the plurality of pins comprises: a first plurality of pins contacting the first plurality of signal conductors and extending away from the first side; and a second plurality of pins contacting the second plurality of signal conductors and extending away from the second side and from the first plurality of pins.

A further embodiment of the foregoing sensor signal connector, further comprising a cylindrical or frustoconical shroud extending from the connector cable and surrounding the modular plug.

A further embodiment of the foregoing sensor signal connector, further comprising a resilient grip abutting the connector cable, the shroud, and the wire guide.

A further embodiment of the foregoing sensor signal connector, wherein the modular plug includes a plurality of alignment features configured to prevent misalignment of the connector plug with an improper receptacle, or at a nonfunctional angle.

A further embodiment of the foregoing sensor signal connector, wherein the alignment features include a keyway flared inboard of a distal end of the modular plug.

A further embodiment of the foregoing sensor signal connector, wherein the modular plug includes an angular cutout.

A further embodiment of the foregoing sensor signal connector, wherein the modular plug has a oval or elliptical cross-section in at least one location, the oval or elliptical cross-section having a major axis parallel to the first and second planes.

A further embodiment of the foregoing sensor signal connector, wherein the major axis has a width of at most 0.5 inches.

A further embodiment of the foregoing sensor signal connector, wherein the elliptical cross-section of the modular plug is further defined by a minor axis orthogonal to the major axis, the minor axis having a width of at most 0.37 inches.

A further embodiment of the foregoing sensor signal connector, wherein the plurality of pins consists of eight pins, each of the 8 pins being electrically connected to one and only one among the first or second pluralities of signal conductors.

A further embodiment of the foregoing sensor signal connector, wherein one of the plurality of pins is a U-shaped double pin.

A further embodiment of the foregoing sensor signal connector, wherein the modular plug exposes the plurality of pins through a plurality of slots, each slot aligned with one of the plurality of pins.

A multi-function signal connector configured to receive both analog and digital sensor signals, the multi-function signal connector comprising: an oval socket disposed about a receptacle axis and defining a receptacle space; a rigid contact support disposed within the oval socket, the rigid contact support including a top shelf and a bottom shelf; a first plurality of electrical contacts disposed along the top shelf, and angled from the top shelf towards the bottom shelf; and a second plurality of electrical contacts disposed along the bottom shelf, and angled from the bottom shelf towards the top shelf.

The multi-function signal connector of the preceding paragraph can optionally include, additionally and/or alternatively, any one or more of the following features, configurations and/or additional components:

A further embodiment of the foregoing multi-function signal connector, wherein the oval socket is attached to the rigid contact support via a snap connection.

A further embodiment of the foregoing multi-function signal connector, wherein the first plurality of electrical contacts is configured to receive an analog signal and routed to an analog signal output, while the second plurality of electrical contacts is configured to receive a digital signal and routed to a digital signal output.

A further embodiment of the foregoing multi-function signal connector, further comprising a substantially cylindrical or frustoconical resilient outer shell surrounding the oval socket.

A further embodiment of the foregoing multi-function signal connector, wherein the oval socket has a substantially circular outer cross-section, and a substantially oval or elliptical inner cross-section.

A further embodiment of the foregoing multi-function signal connector, wherein the oval socket includes a plurality of alignment features.

A further embodiment of the foregoing multi-function signal connector, wherein the alignment features comprise alignment shelves at substantially opposite ends of the receptacle space.

A further embodiment of the foregoing multi-function signal connector, wherein the alignment features further comprise a snap key disposed symmetrically between the alignment shelves.

A further embodiment of the foregoing multi-function signal connector, wherein the snap key separates a subset of the second plurality of electrical contacts from the remainder of the second plurality of electrical contacts.

A further embodiment of the foregoing multi-function signal connector, wherein the snap key includes a flared portion of resilient material.

A further embodiment of the foregoing multi-function signal connector, wherein the entirety of the oval socket is formed of the resilient material.

A further embodiment of the foregoing multi-function signal connector, further comprising a common circuit board to which both the first and second pluralities of electrical contacts are anchored.

A further embodiment of the foregoing multi-function signal connector, wherein the first plurality of electrical contacts are anchored to an opposite side of the common circuit board from the second plurality of electrical contacts.

A further embodiment of the foregoing multi-function signal connector, wherein the first and second pluralities of electrical contacts are spring loaded.

A differential pressure transducer assembly comprising: a flowpath element comprising: a channel wall defining a fluid channel connecting a fluid inlet to a fluid outlet, the fluid channel defining a centerline axis of the differential pressure transducer assembly; a lateral plate extending perpendicularly outward from opposite sides of the channel wall; and a plurality of protrusions cantilevered from the lateral plate, each protrusion of the plurality of protrusions comprising: a beam extending normal to the lateral plate and extending parallel to a beam axis; and a barb disposed at a distal end of the beam; and a structural housing comprising: a base; a side wall extending from the base to define a recess conforming to an outer periphery of the lateral plate; and a plurality of receptacles adapted to receive respective protrusions of the plurality of protrusions, each receptacle of the plurality of receptacles comprising: a pocket open along the beam axis; and an undercut extending perpendicularly to the beam axis into the structural housing from the pocket; wherein each protrusion of the plurality of protrusions is insertable along the beam axis into respective receptacles.

The differential pressure transducer assembly of the preceding paragraph can optionally include, additionally and/or alternatively, any one or more of the following features, configurations and/or additional components:

A further embodiment of the foregoing differential pressure transducer assembly, wherein the pocket of each receptacle is disposed interior to the side wall.

A further embodiment of the foregoing differential pressure transducer assembly, wherein the undercut of each receptacle extends outward within the base relative to the centerline axis and under the side wall.

A further embodiment of the foregoing differential pressure transducer assembly, wherein each receptacle of the plurality of receptacles comprises: an opening extending through a first face of the base to intersect the undercut of each receptacle, wherein the side wall extends from a second face of the base opposite the first face.

A further embodiment of the foregoing differential pressure transducer assembly, wherein the pocket of each receptacle receives the beam of a respective protrusion, and wherein the undercut of each receptacle receives the barb of the respective protrusion via the pocket.

A further embodiment of the foregoing differential pressure transducer assembly, wherein the barb of each protrusion includes a lip surface extending perpendicularly from the beam that opposes a retention surface of the undercut of each receptacle.

A further embodiment of the foregoing differential pressure transducer assembly, wherein the barb of each protrusion includes a tapered surface that is oblique to the beam axis.

A further embodiment of the foregoing differential pressure transducer assembly, wherein the structural housing further comprises: a plurality of shelf faces formed by the side wall, wherein each shelf face is oblique to the beam axis and oriented to abut a respective tapered surface of the plurality of protrusions during assembly of the flowpath element to the structural housing.

A further embodiment of the foregoing differential pressure transducer assembly, wherein each shelf face of the plurality of shelf faces is positioned at a distal end of the side wall opposite the base.

A further embodiment of the foregoing differential pressure transducer assembly, wherein each shelf face of the plurality of shelf faces is positioned at a distal end of the side wall opposite the base.

A further embodiment of the foregoing differential pressure transducer assembly, wherein each barb of the plurality of protrusions is outward facing relative to the centerline axis of the flowpath element, and wherein each pocket of the plurality of receptacles is interior of the side wall.

A further embodiment of the foregoing differential pressure transducer assembly, wherein at least one protrusion of the plurality of protrusions includes a rib extending along a first face of the beam, and wherein the barb extends from a second face of the beam opposite the first face of the beam.

A further embodiment of the foregoing differential pressure transducer assembly, wherein the plurality of protrusions includes a first protrusion and a second protrusion, and wherein each of the first protrusion and the second protrusion includes a first rib extending from the lateral plate to the distal end of respective beams.

A further embodiment of the foregoing differential pressure transducer assembly, wherein the plurality of protrusions includes a third protrusion and a fourth protrusion, and wherein each of the third protrusion and the fourth protrusion includes a second rib extending from the lateral plate and terminating between the lateral plate and the distal tip of respective beams.

A further embodiment of the foregoing differential pressure transducer assembly, wherein a cross-section of each pocket normal to the beam axis conforms to cross sections of respective beams and ribs taken normal to the beam axis.

A further embodiment of the foregoing differential pressure transducer assembly, further comprising: a sensor housed within a cavity defined by the flowpath element; and a connector cable extending along the centerline axis between the flowpath element and the structural housing, wherein the connector cable comprises: a plurality of conductors that electrically connects the connector cable to the sensor; and a protective cover circumscribing the plurality of conductors.

A further embodiment of the foregoing differential pressure transducer assembly, wherein the flowpath element includes a cable pedestal extending from the channel wall parallel to the beam axis that engages the protective cover of the connector cable.

A further embodiment of the foregoing differential pressure transducer assembly, wherein the structural housing includes a saddle formed by the base that engages the protective cover of the connector cable.

A further embodiment of the foregoing differential pressure transducer assembly, wherein the cable pedestal includes a terminal surface that conforms to the protective cover of the connector cable.

A further embodiment of the foregoing differential pressure transducer assembly, wherein the saddle conforms to the protective cover of the connector cable.

A further embodiment of the foregoing differential pressure transducer assembly, wherein the cable pedestal and the saddle interfere with the protective cover of the connector cable such that the protective cover of the connector cable biases the flowpath element toward the structural housing to engage abutting surfaces of the barb and the undercut.

A further embodiment of the foregoing differential pressure transducer assembly, wherein the cable pedestal is disposed between two protrusions of the plurality of protrusions, and wherein the saddle is disposed between two receptacles of the plurality of receptacles.

A differential pressure transducer assembly comprising: a flowpath element comprising: a channel wall defining a fluid channel connecting a fluid inlet to a fluid outlet, the fluid channel defining a centerline axis of the differential pressure transducer assembly; a stopcock comprising: a cylindrical body; and a first semiannular collar segment subtending a first sector of the cylindrical body; a structural housing mated to and supporting the flowpath element; a front cover mated to and cooperating with a rear cover to surround the structural housing and the flowpath element; a pair of cantilevered supports extending from the front cover or the rear cover to elastically engage the structural housing biasing the structural housing and the flowpath element into engagement with the stopcock; and a support pedestal extending from the rear cover that defines a semicylindrical channel and a first retaining slot, wherein the semicylindrical channel receives the flowpath element and the stopcock, and wherein first the retaining slot receives the first semiannular rib, and wherein abutting surfaces of the first semiannular rib and the first retaining slot restrain the stopcock, the flowpath element, and the structural housing against the pair of cantilevered supports.

The differential pressure transducer of the preceding paragraph can optionally include, additionally and/or alternatively, any one or more of the following features, configurations and/or additional components:

A further embodiment of the foregoing differential pressure transducer, wherein the pair of cantilevered supports includes a pair of longitudinal ribs, each longitudinal rib extending along one of the cantilevered supports opposite the structural housing.

A further embodiment of the foregoing differential pressure transducer, wherein a cross-sectional area of the pair of longitudinal ribs decreases towards a free end of the pair of longitudinal ribs.

A further embodiment of the foregoing differential pressure transducer, wherein the cross-sectional area of the pair of longitudinal ribs decreases linearly towards the free end of the pair of longitudinal ribs.

A further embodiment of the foregoing differential pressure transducer, wherein the pair of cantilevered supports includes a pair of side gussets, each side gusset extending from one of the cantilevered supports to the front cover or the rear cover.

A further embodiment of the foregoing differential pressure transducer, wherein the first retaining slot of the pedestal is semiannular, and wherein circumferentially abutting surfaces of the first retaining slot and the semiannualar rib restrain rotation of the stopcock about the centerline axis.

A further embodiment of the foregoing differential pressure transducer, wherein the stopcock comprises a second semiannular collar segment subtending a second sector of the cylindrical body that is spaced circumferentially from the first semiannular collar segment, and wherein the pedestal comprises a second retaining slot that receives the second semiannular collar segment, and wherein abutting surfaces of the second retaining slot and the second semiannular collar segment restrain the stopcock, the flowpath element, and the structural housing against the pair of cantilevered supports.

A further embodiment of the foregoing differential pressure transducer, wherein the first retaining slot and the second retaining slot of the pedestal are semiannular, and wherein circumferentially abutting surfaces of the first retaining slot and the first semiannular collar segment and circumferentially abutting surfaces of the second retaining slot and the second semiannular collar segment restrain rotation of the stopcock about the centerline axis.

A further embodiment of the foregoing differential pressure transducer, where the first semiannular collar segment coincides with the second semiannular collar segment along the centerline axis.

A further embodiment of the foregoing differential pressure transducer, wherein the pair of cantilevered supports includes a pair of longitudinal ribs, each longitudinal rib extending along one of the cantilevered supports opposite the structural housing; wherein the first retaining slot of the pedestal is semiannular; and wherein circumferentially abutting surfaces of the first retaining slot and the semiannular collar segment restrain rotation of the stopcock about the centerline axis.

A further embodiment of the foregoing differential pressure transducer, wherein the stopcock comprises a second semiannular collar segment subtending a second sector of the cylindrical body that is spaced circumferentially from the first semiannular collar segment; and wherein the pedestal comprises a second retaining slot that receives the second semiannular collar segment; and wherein abutting surfaces of the second retaining slot and the second semiannular collar segment restrain the stopcock, the flowpath element, and the structural housing against the pair of cantilevered supports.

A further embodiment of the foregoing differential pressure transducer, wherein the first retaining slot and the second retaining slot of the pedestal are semiannular, and wherein circumferentially abutting surfaces of the first retaining slot and the first semiannular collar segment and circumferentially abutting surfaces of the second retaining slot and the second semiannular collar segment restrain rotation of the stopcock about the centerline axis.

A further embodiment of the foregoing differential pressure transducer, where the first semiannular collar segment coincides with the second semiannular collar segment along the centerline axis.

A further embodiment of the foregoing differential pressure transducer, wherein the structural housing includes a snap slot; and wherein the rear cover includes a snap attachment comprising a plurality of barbed flanges that engage the snap slot to restrain the structural housing in a first direction normal to the base and permit sliding motion of the structural housing relative to the rear casing in a second direction parallel to the centerline axis.

A differential pressure transducer assembly comprising: a flowpath element comprising: a channel wall defining a fluid channel connecting a fluid inlet to a fluid outlet, the fluid channel defining a centerline axis of the differential pressure transducer assembly; a lateral plate extending perpendicularly outward from opposite sides of the channel wall; and a plurality of protrusions cantilevered from the lateral plate, each protrusion of the plurality of protrusions comprising: a beam extending normal to the lateral plate and extending parallel to a beam axis; and a barb disposed at a distal end of the beam; a structural housing comprising: a base; a side wall extending from the base to define a recess conforming to an outer periphery of the lateral plate; and a plurality of receptacles adapted to receive respective protrusions of the plurality of protrusions, each receptacle of the plurality of receptacles comprising: a pocket open along the beam axis; and an undercut extending perpendicularly to the beam axis into the structural housing from the pocket, wherein each protrusion of the plurality of protrusions is insertable along the beam axis into respective receptacles along the beam axis; a stopcock comprising: a cylindrical body; and a first semiannular collar segment subtending a first sector of the cylindrical body; a front cover mated to and cooperating with a rear cover to surround the structural housing and the flowpath element; a pair of cantilevered supports extending from the front cover or the rear cover to elastically engage the structural housing biasing the structural housing and the flowpath element into engagement with the stopcock; and a support pedestal extending from the rear cover that defines a semicylindrical channel and a first retaining slot, wherein the semicylindrical channel receives the flowpath element and the stopcock, and wherein first the retaining slot receives the first semiannular collar segment, and wherein abutting surfaces of the first semiannular collar segment and the first retaining slot restrain the stopcock, the flowpath element and the structural housing against the pair of cantilevered supports.

The differential pressure transducer assembly of the preceding paragraph can optionally include, additionally and/or alternatively, any one or more of the following features, configurations and/or additional components:

A further embodiment of the foregoing differential pressure transducer assembly, wherein each barb of the plurality of protrusions is outward facing relative to the centerline axis of the flowpath element, and wherein each pocket of the plurality of receptacles is interior of the side wall.

A further embodiment of the foregoing differential pressure transducer assembly, wherein at least one protrusion of the plurality of protrusions includes a rib extending along a first face of the beam, and wherein the barb extends from a second face of the beam opposite the first face of the beam.

A further embodiment of the foregoing differential pressure transducer assembly, wherein the plurality of protrusions includes a first protrusion and a second protrusion, and wherein each of the first protrusion and the second protrusion includes a first rib extending from the lateral plate to the distal end of respective beams.

A further embodiment of the foregoing differential pressure transducer assembly, wherein the plurality of protrusions includes a third protrusion and a fourth protrusion, and wherein each of the third protrusion and the fourth protrusion includes a second rib extending from the lateral plate and terminating between the lateral plate and the distal tip of respective beams.

A further embodiment of the foregoing differential pressure transducer assembly, further comprising: a sensor housed within a cavity defined by the flowpath element; and a connector cable extending along the centerline axis between the flowpath element and the structural housing, wherein the connector cable comprises: a plurality of conductors that electrically connects the connector cable to the sensor; and a protective cover that circumscribes the plurality of conductors.

A further embodiment of the foregoing differential pressure transducer assembly, wherein the flowpath element includes a cable pedestal extending from the channel wall parallel to the beam axis that engages the protective cover of the connector cable, and wherein the structural housing includes a saddle formed by the base that engages the protective cover of the connector cable.

A further embodiment of the foregoing differential pressure transducer assembly, wherein the cable pedestal and the saddle interfere with the protective cover of the connector cable such that the protective cover of the connector cable biases the flowpath element toward the structural housing to engage abutting surfaces of the barb and the undercut.

A further embodiment of the foregoing differential pressure transducer assembly, wherein the cable pedestal is disposed between two protrusions of the plurality of protrusions, and wherein the saddle is disposed between two receptacles of the plurality of receptacles.

A further embodiment of the foregoing differential pressure transducer assembly, wherein the structural housing includes a snap slot; and wherein the rear cover includes a snap attachment comprising a plurality of barbed flanges that engage the snap slot to restrain the structural housing a first direction normal to the base and permit sliding motion of the structural housing relative to the rear casing in a second direction parallel to the centerline axis.

A further embodiment of the foregoing differential pressure transducer assembly, wherein the pair of cantilevered supports includes a pair of longitudinal ribs, each longitudinal rib extending along one of the cantilevered supports opposite the structural housing.

A further embodiment of the foregoing differential pressure transducer assembly, wherein a cross-sectional area of the pair of longitudinal ribs decreases towards a free end of the pair of longitudinal ribs.

A further embodiment of the foregoing differential pressure transducer assembly, wherein the cross-sectional area of the pair of longitudinal ribs decreases linearly towards the free end of the pair of longitudinal ribs.

A further embodiment of the foregoing differential pressure transducer assembly, wherein the pair of cantilevered supports includes a pair of side gussets, each side gusset extending from one of the cantilevered supports to the front cover or the rear cover.

A further embodiment of the foregoing differential pressure transducer assembly, wherein the pair of cantilevered supports includes a pair of longitudinal ribs, each longitudinal rib extending along one of the cantilevered supports opposite the structural housing; wherein the first retaining slot of the pedestal is semiannular; and wherein circumferentially abutting surfaces of the first retaining slot and the semiannualar collar segment restrain rotation of the stopcock about the centerline axis.

A further embodiment of the foregoing differential pressure transducer assembly, wherein the stopcock comprises a second semiannular collar segment subtending a second sector of the cylindrical body that is spaced circumferentially from the first semiannular collar segment; and wherein the pedestal comprises a second retaining slot that receives the second semiannular collar segment; and wherein abutting surfaces of the second retaining slot and the second semiannular collar segment restrain the stopcock, the flowpath element, and the structural housing against the pair of cantilevered supports.

A further embodiment of the foregoing differential pressure transducer assembly, wherein the first retaining slot and the second retaining slot of the pedestal are semiannular, and wherein circumferentially abutting surfaces of the first retaining slot and the first semiannular collar segment and circumferentially abutting surfaces of the second retaining slot and the second semiannular collar segment restrain rotation of the stopcock about the centerline axis.

A further embodiment of the foregoing differential pressure transducer assembly, wherein the first semiannular collar segment coincides with the second semiannular collar segment along the centerline axis.

Any relative terms or terms of degree used herein, such as “substantially”, “essentially”, “generally”, “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, alignment or shape variations induced by thermal, rotational or vibrational operational conditions, and the like.

While this disclosure has been provided with reference to particular illustrative examples, it will be understood by those skilled in the art that various changes may be made and equivalents may be substituted for elements thereof without departing from the scope or spirit of this disclosure. In addition, many modifications may be made to adapt a particular situation or material to the teachings of this disclosure without departing from the essential scope thereof.

1. A dual differential pressure transducer assembly comprising:
an intravenous (IV) fluid inlet configured to receive fluid from an IV bag;
an IV fluid outlet configured to supply the fluid to a patient IV needle;
a flowpath element defining a fluid channel connecting the IV fluid inlet to the IV fluid outlet, the fluid channel defining a centerline axis of the dual differential pressure transducer assembly;
a first sensor cavity disposed alongside the fluid channel and fluidly connected to the fluid channel via a first cutout through the flowpath element, the first cutout extending transverse to the centerline axis;
a second sensor cavity disposed alongside the fluid channel, parallel with the first sensor cavity, and fluidly connected to the fluid channel via a second cutout through the flowpath element, the second cutout extending transverse to the centerline axis, and axially aligned but angularly offset from the first cutout, relative to the centerline axis;
a first differential pressure sensor having a first sensor face abutting the first sensor cavity and exposed to the fluid channel via the first cutout;
a second differential pressure sensor having a second sensor face abutting the second sensor cavity and exposed to the fluid channel via the second cutout; and
separate signal conductors connected to the first and second differential pressure sensors, respectively.

2. The dual differential pressure transducer assembly of claim 1, wherein the first differential pressure sensor is an analog sensor, and the second differential pressure sensor is a digital sensor.

3. The dual differential pressure transducer assembly of claim 2, wherein the separate signal conductors comprise:
a first plurality of signal conductors disposed to carry analog electrical signals from the first differential pressure sensor; and
a second plurality of signal conductors disposed to carry digital signals from the second differential pressure sensor,
wherein the first and second pluralities of signal conductors are arranged in a common plane alongside the first and second sensors, and enclosed together with the first and second differential pressure sensors within a common housing.

4. The dual differential pressure transducer assembly of claim 3, wherein the common housing surrounds the first differential pressure sensor, the second differential pressure sensor, and at least a portion of the fluid channel aligned with the first and second cutouts.

5. The dual differential pressure transducer assembly of claim 1, wherein the first and second cutouts are separated by an angle less than 180° or greater than 90°, relative to the centerline axis.

6. The dual differential pressure transducer assembly of claim 1, wherein the fluid channel has a diameter between 1 and 8 mm at the axial location of the first and second cutouts.

7. The dual differential pressure transducer assembly of claim 1, wherein a cross-sectional area of the fluid channel through planes orthogonal to the centerline axis is narrowest at a location upstream of the first and second cutouts.

8. The dual differential pressure transducer assembly of claim 1, wherein the first and second cutouts intersect the fluid channel at first and second channel apertures, and the first and second channel apertures are elongate apertures with major dimensions parallel to the centerline axis.

9. The dual differential pressure transducer assembly of claim 8, wherein the first and second channel apertures each have a circumferential width relative to the centerline axis that is greater than 0.005 inches (0.133 mm) or less than 25% of the total circumference of the channel at locations of the first and second channel apertures.

10. The dual differential pressure transducer assembly of claim 9, wherein the first and second channel apertures each have an axial length, relative to the centerline axis, of at least 0.005 inches, and less than an axial extent of sealing surfaces of the first and second differential pressure sensors.

11. The dual differential pressure transducer assembly of claim 9, wherein the first and second channel apertures each have an axial length, relative to the centerline axis, of between 0.07 and 0.08 inches.

12. The dual differential pressure transducer assembly of claim 8, wherein the first and second channel apertures each have an angular extent less than 5° with respect to the centerline axis.

13. The dual differential pressure transducer assembly of claim 8, wherein the first and second cutouts each:
widen as a function of radial position with respect to the centerline axis, from narrowest at the first and second channel apertures, respectively, to widest at the first and second sensor faces, respectively; and
are defined in part by walls extending from the fluid channel to the first and second differential pressure sensors, respectively, and wherein the walls are parallel to but offset from each other and the centerline axis.

14. The dual differential pressure transducer assembly of claim 1, wherein at least one of the first and second sensor faces is defined by a deflecting gel diaphragm.

15. The dual differential pressure transducer assembly of claim 1, further comprising a stopcock disposed along the fluid channel, downstream of the first and second cutouts, and actuatable between multiple valve states, including:
a first state fluidly separating the IV fluid inlet from the IV fluid outlet; and
a second state fluidly connecting the IV fluid inlet to the IV fluid outlet.

16. The dual differential pressure transducer assembly of claim 1, further comprising a flush tab disposed alongside a wall within the fluid channel and selectively permit flow past the wall, from upstream to downstream through the fluid channel, wherein the flush tab is disposed upstream of the first and second cutouts.

17. The dual differential pressure transducer assembly of claim 1, wherein the dual differential pressure transducer assembly is sterilized.

18. The dual differential pressure transducer assembly of claim 1, further comprising a connector cable terminating at a connector plug and surrounding the separate signal conductors from the first and second differential pressure sensors to the connector plug.

19. The dual differential pressure transducer assembly of claim 18, wherein the connector plug comprises:
a plug body disposed to be inserted in a receiving receptacle;
a first plurality of pins electrically connected to first differential pressure sensor via a first subset of the separate signal conductors; and
a second plurality of pins electrically connected to the second differential pressure sensor via the second subset of the separate signal conductors separate from the first subset of the separate signal conductors;
wherein the first and second pluralities of pins are disposed on opposite sides of the plug body.

20. A differential pressure transducer assembly comprising:
a flowpath element comprising:
a channel wall defining a fluid channel connecting a fluid inlet to a fluid outlet, the fluid channel defining a centerline axis of the differential pressure transducer assembly;
a lateral plate extending perpendicularly outward from opposite sides of the channel wall; and
a plurality of protrusions cantilevered from the lateral plate, each protrusion of the plurality of protrusions comprising:
a beam extending normal to the lateral plate and extending parallel to a beam axis; and
a barb disposed at a distal end of the beam;
a structural housing comprising:
a base;
a side wall extending from the base to define a recess conforming to an outer periphery of the lateral plate; and
a plurality of receptacles adapted to receive respective protrusions of the plurality of protrusions, each receptacle of the plurality of receptacles comprising:
a pocket open along the beam axis; and
an undercut extending perpendicularly to the beam axis into the structural housing from the pocket,
wherein each protrusion of the plurality of protrusions is insertable along the beam axis into respective receptacles along the beam axis;
a stopcock comprising:
a cylindrical body; and
a first semiannular collar segment subtending a first sector of the cylindrical body;
a front cover mated to and cooperating with a rear cover to surround the structural housing and the flowpath element;
a pair of cantilevered supports extending from the front cover or the rear cover to elastically engage the structural housing biasing the structural housing and the flowpath element into engagement with the stopcock; and
a support pedestal extending from the rear cover that defines a semicylindrical channel and a first retaining slot, wherein the semicylindrical channel receives the flowpath element and the stopcock, and wherein first the retaining slot receives the first semiannular collar segment, and wherein abutting surfaces of the first semiannular collar segment and the first retaining slot restrain the stopcock, the flowpath element and the structural housing against the pair of cantilevered supports.
